SEQUENCE LISTING (44 sequences)

SEQ ID NO: 1 - DNA, 37 bp, Artificial sequence
  Synthetic
gtaataggatcccaaccatagttcatatcaagtggaa

SEQ ID NO: 2 - DNA, 20 bp, Artificial sequence
  Synthetic
aagttgatgagcggcaacag

SEQ ID NO: 3 - DNA, 45 bp, Artificial sequence
  Synthetic
ctgttgccgctcatcaacttcaaaatgtgattaaaacaaatggcc

SEQ ID NO: 4 - DNA, 32 bp, Artificial sequence
  Synthetic
ttagtagagctcgacttgcatgcaccacaaat

SEQ ID NO: 5 - DNA, 22 bp, Artificial sequence
  Synthetic
tgctcaaaacgcacatgtttca

SEQ ID NO: 6 - DNA, 20 bp, Artificial sequence
  Synthetic
actcgtgctcgtgaaccaat

SEQ ID NO: 7 - DNA, 26 bp, Artificial sequence
  Synthetic
gaacactatgttccatcttaggaaaa

SEQ ID NO: 8 - DNA, 37 bp, Artificial sequence
  Synthetic
gtaataggatccggtagtattagctaatttaggaaca

SEQ ID NO: 9 - DNA, 20 bp, Artificial sequence
  Synthetic
taatgcaacgattggtcttg

SEQ ID NO: 10 - DNA, 40 bp, Artificial sequence
  Synthetic
caagaccaatcgttgcattactctacaagcaggaacaaca

SEQ ID NO: 11 - DNA, 32 bp, Artificial sequence
  Synthetic
ttagtagaattcaatccttatttccggtagct

SEQ ID NO: 12 - DNA, 23 bp, Artificial sequence
  Synthetic
gttgttaacgaatctgttgatca

SEQ ID NO: 13 - DNA, 22 bp, Artificial sequence
  Synthetic
atcgtttaaaaattgccattgc

SEQ ID NO: 14 - DNA, 20 bp, Artificial sequence
  Synthetic
tcaacggtagataatgacga

SEQ ID NO: 15 - DNA, 20 bp, Artificial sequence
  Synthetic
agatgcagaacacggtggtc

SEQ ID NO: 16 - DNA, 22 bp, Artificial sequence
  Synthetic
gtccaatagtcattcctgcacc

SEQ ID NO: 17 - DNA, 19 bp, Artificial sequence
  Synthetic
tactcaaagaaggcttacg

SEQ ID NO: 18 - DNA, 19 bp, Artificial sequence
  Synthetic
attaactacggcttgaacc

SEQ ID NO: 19 - DNA, 22 bp, Artificial sequence
  Synthetic
ggcaaccgttgtgatggttatc

SEQ ID NO: 20 - DNA, 21 bp, Artificial sequence
  Synthetic
accttgcaaagtttcttgggc

SEQ ID NO: 21 - DNA, 20 bp, Artificial sequence
  Synthetic
taccggtcttcaccacttgg

SEQ ID NO: 22 - DNA, 20 bp, Artificial sequence
  Synthetic
gctgcgtattctgcaaggtg

SEQ ID NO: 23 - DNA, 34 bp, Artificial sequence
  Synthetic
gtaataggatcctcacattgattttgccgttact

SEQ ID NO: 24 - DNA, 20 bp, Artificial sequence
  Synthetic
tctttgccaccaacatcttt

SEQ ID NO: 25 - DNA, 42 bp, Artificial sequence
  Synthetic
aaagatgttggtggcaaagaacatcagttaatggacaagtgc

SEQ ID NO: 26 - DNA, 32 bp, Artificial sequence
  Synthetic
ttagtagagctctctagcatcattacggctgt

SEQ ID NO: 27 - DNA, 27 bp, Artificial sequence
  Synthetic
caggttaaagagtttaaatcacaaaca

SEQ ID NO: 28 - DNA, 20 bp, Artificial sequence
  Synthetic
cacgagcacttgcaacaaat

SEQ ID NO: 29 - DNA, 25 bp, Artificial sequence
  Synthetic
tgaactggacattagattcagacga

SEQ ID NO: 30 - DNA, 20 bp, Artificial sequence
  Synthetic
atcttcggtgttcactgggg

SEQ ID NO: 31 - DNA, 20 bp, Artificial sequence
  Synthetic
aaacaaccccgatttgtgcg

SEQ ID NO: 32 - DNA, 35 bp, Artificial sequence
  Synthetic
gtaataggatccaagtcagtagatgcaaaatatga

SEQ ID NO: 33 - DNA, 20 bp, Artificial sequence
  Synthetic
gtaggcaccttcaatttgat

SEQ ID NO: 34 - DNA, 43 bp, Artificial sequence
  Synthetic
atcaaattgaaggtgcctactcacttaagagacttcctaagga

SEQ ID NO: 35 - DNA, 32 bp, Artificial sequence
  Synthetic
ttagtagaattcagtccgcttgtcatcatagt

SEQ ID NO: 36 - DNA, 21 bp, Artificial sequence
  Synthetic
aagggggttcaatgactcaaa

SEQ ID NO: 37 - DNA, 27 bp, Artificial sequence
  Synthetic
gcttcatacaaaaattcagatttgaca

SEQ ID NO: 38 - DNA, 25 bp, Artificial sequence
  Synthetic
ttgttaaaggtgaagtaaaggtagg

SEQ ID NO: 39 - DNA, 20 bp, Artificial sequence
  Synthetic
tgcttggtccttagctggtg

SEQ ID NO: 40 - DNA, 20 bp, Artificial sequence
  Synthetic
caatgccgcagtaaccgaag

SEQ ID NO: 41 - DNA, 20 bp, Artificial sequence
  Synthetic
tcccagatacctgaaacgcc

SEQ ID NO: 42 - DNA, 20 bp, Artificial sequence
  Synthetic
aaatgaagtttggccaggcg

SEQ ID NO: 43 - DNA, 21 bp, Artificial sequence
  Synthetic
ccgcgttgcagatacatcaac

SEQ ID NO: 44 - DNA, 21 bp, Artificial sequence
  Synthetic
tcacaacccacgctttattgg

(19) United States
(12) Patent Application Publication
(10) Pub. No.: US 2020/0222474 A1
(43) Pub. Date: Jul. 16, 2020

(54) COMPOSITIONS AND METHODS FOR INCREASING PHYTOCHEMICAL BIOAVAILABLITY AND BIOACTIVITY

(71) Applicants: North Carolina State University, Raleigh, NC (US); Danmarks Tekniske Universitet, Kgs. Lungby (DK)

(72) Inventors: Mia C. Theilmann, Copenhagen Ø (DK); Yong Jun Goh, Apex, NC (US); Rodolphe Barrangou, Raleigh, NC (US); Maher Abou Hachem, Espergærde (DK)

(21) Appl. No.: 16/633,427

(22) PCT Filed: Jul. 23, 2018
(86) PCT No.: PCT/US2018/043305

§ 371 Date: Jan. 23, 2020

Related U.S. Application Data
(60) Provisional application No. 62/536,209, filed on Jul. 24, 2017.

(51) Int. Cl. A61K 35/747; A61K 31/7034; A23L 33/135; A23L 33/105; A61K 38/47; A61K 35/745

(52) U.S. Cl. CPC A61K 35/747; A61K 31/7034; A23L 33/135; A61K 2035/115; A61K 38/47; A61K 35/745; A23L 33/105

(57) ABSTRACT

The present disclosure relates to the field of microbiota research and therapy. In particular, the present disclosure pro-vides compositions and methods for increasing bioavailability of phytochemicals using probiotic bacteria. Compositions and methods described herein include combinations of probiotic bacteria and prebiotic plant glycosides, wherein the probiotic bacteria are capable of converting the prebiotic plant glycosides into aglycones with increased bioavailability.

### CROSS-REFERENCE TO RELATED APPLICATIONS

This application claims the benefit of the earlier filing date of U.S. Provisional Application No. 62/536,209, filed Jul. 24, 2017, which is incorporated herein by reference in its entirety.

### SEQUENCE LISTING

The sequence listing is filed with the application in electronic format only and is incorporated by reference herein. The sequence listing text file “030871-9069 Sequence Listing.txt” was created on Jul. 23, 2018, and is 8.257 bytes in size.

### TECHNICAL FIELD

The present disclosure relates to the field of microbiota research and therapy. In particular, the present disclosure provides compositions and methods for increasing bioavailability of phytochemicals using probiotic bacteria. Compositions and methods described herein include combinations of probiotic bacteria and prebiotic plant glycosides, wherein the probiotic bacteria are capable of converting the prebiotic plant glycosides into aglycones with increased bioavailability.

### BACKGROUND

Xenobiotic phytochemicals occur in various food sources, such as berries, fruits, nuts, vegetables, and also in beverages such as wine and tea. These compounds typically exist as glyco-conjugates to facilitate storage and solubility, and to modulate biological activity. Several phytochemicals (e.g., some phenolic and polyphenolic compounds) exhibit beneficial health effects via anti-inflammatory, antiestrogenic, cardioprotective, anticarcinogenic, chemopreventative, neuroprotective, antimicrobial or antioxidants properties. These biological activities vary depending on the glyco-conjugation of the phytochemical. In some cases, probiotic bacteria (e.g., strains from lactobacilli) have been reported to interact with these glycosylated phytochemicals, or plant glycosides (PGs), but the role of these probiotic bacteria and the nature of their biothemical interactions with PGs is not fully understood. A significant proportion of the thousands of diet-derived known phytochemicals exhibit positive health effects in humans. However, it is often the case that phytochemicals occur as glyco-conjugates, and thus exhibit lower bioactivity and bioavailability than their aglycone derivatives, which are smaller in size and typically less polar. The deglycosylation of PGs may be a factor in modulating their biological activity. Recently, the health-impact of human gut microbiota (HGM)-mediated biotransformation of drug and diet-derived xenobiotics, including phytochemicals, has gained considerable interest, but knowledge of the metabolic mechanisms and the therapeutic potential of the HGM are significantly limited. Thus, there is a need for a greater understanding of the interaction of various probiotic bacteria in the HGM and their therapeutic potential for enhancing the bioavailability and bioactivity of beneficial compounds.

### SUMMARY

The present disclosure is directed to compositions that include a probiotic bacterial strain, a prebiotic plant glycoside, and a physiologically acceptable carrier and/or excipient, wherein the probiotic bacterial strain is capable of converting the prebiotic plant glycoside into a bioactive aglycone, or derivative thereof.

The present disclosure is also directed to nutritional supplements that include said compositions.

The present disclosure is also directed to methods for providing a dietary supplement to a subject. The methods include administering to the subject said composition or said nutritional supplement.

The present disclosure is also directed to methods of supplementing a fermented dairy product. The methods include mixing said composition or said nutritional supplement with the fermented dairy product.

The present disclosure is also directed to methods of treating a condition in a subject in need thereof. The methods include administering said compositions to the subject to treat the condition thereby treating the condition.

### BRIEF DESCRIPTION OF THE DRAWINGS

FIGS. 1A-1B include representative data from experiments involving growth of Lactobacillus acidophilus NCFM on plant glycosides. FIG. 1A shows structures and common sources of plant glycosides substrates, as described herein. FIG. 1B is a representative graph depicting plant glycoside utilization analyzed by mass spectrometry and the growth as the maximum OD₆₀₀.

FIGS. 2A-2B include representative transcriptional profiles illustrating the conservation of plant glycoside utilization loci. FIG. 2A is a representative graph showing the top upregulated locus in L. acidophilus NCFM on three plant glycosides, which includes a transcriptional regulator (LBA0724), a PTS EIIBCA transporter (LBA0725), and a phospho-β-glucosidase (P-Bgl) of glycoside hydrolase family 1 (GH1) (LBA0726), FIG. 2B is a representative graph showing the locus upregulated on amygdalin, which includes a P-Bgl (LBA0225), a PTS EIIC transporter (LBA0227), and a hypothetical protein.

FIGS. 3A-3J are representative graphs showing growth analyses of various deletion mutants. FIGS. 3A, 3C, 3E, 3G, and 3I represent growth analysis of EII PTS transporter mutants, and FIGS. 3B, 3D, 3F, 3H, and 3J represent growth analysis of phospho-β-glucosidase mutants, on esculin (FIGS. 3A-3B), salicin (FIGS. 3C-3D), amygdalin (FIGS. 3E-3F), gentiobiose (FIGS. 3G-3H), and cellobiose (FIGS. 3I-3J).

FIGS. 4A-4B show time-resolved metabolite analysis of L. acidophilus NCFM growing on plant glucosides. FIG. 4A is a representative graph showing the time course depletion of salicin and appearance of its aglycone salicyl alcohol in the culture supernatants visualized as the area under the A_{270 nm} peaks in the UHPLC-qTOF-MS chromatograms. FIG. 4B shows representative graphs of L. acidophilus NCFM growth on an equimolar mixture of salicin, esculin and amygdalin.

FIG. 5 is a representative diagram of a plant glucoside utilization model based on the present disclosure.

FIGS. 6A-6C show time-resolved metabolite analysis of L. acidophilus NCFM growing on plant glucosides. FIG. 6A is a representative graph showing the time course depletion of salicin, FIG. 6B is a representative graph showing the time course depletion of esculin (FIG. 6B), and FIG. 6C is a representative graph showing the time course depletion of amygdalin.

FIG. 7 is a representative graph showing the growth of human gut microbiota commensals from the Bifidobacterium (Bi), Bacteroides (Ba) and Roseburia (R) genera on plant glycosides.

### DETAILED DESCRIPTION

Therapeutically-active plant compounds (e.g., phytochemicals), which frequently occur as glyco-conjugates, are ubiquitous in human diet. Interplay of phytochemicals with the human gut microbiota (HGM) is commensurate to altered microbiota composition and phytochemical bioactivity. Despite the potential health impact of this interplay, the key taxa involved and the underpinning molecular mechanisms remain uncharacterized. Additionally, it has been reported that various phytochemicals from plants exhibit significant bioactivity when tested in in vitro assays, and even in some animal models. However, in many cases, their in vivo efficacy in human is still in question or remains to be established. As would be recognized by one of ordinary skill in the art, the bioactivity and bioavailability of phytochemicals to a subject are dependent on many different factors, including but not limited to, absorption, metabolism, solubility and/or dissolution, permeation, first-pass metabolism and pre-systemic excretion. Therefore, simple ingestion of various phytochemicals is not always sufficient to cause a desired physiological effect, or bioactivity, in a host subject in large part due to the various factors that impede their bioavailability to the host.

Findings of the present disclosure indicate the growth of probiotic bacterial strains, including Lactobacillus acidophilus, on dietary plant glycosides (PG) using specialized uptake and deglycosylation machinery, accompanied with significant upregulation of host-interaction genes in a prebiotic-like transcriptional response. The deglycosylated moieties of PGs that typically possess increased bioactivities as compared to the parent compounds are externalized, rendering them bio-available to the host and other microbiota taxa, The PG utilization loci are largely conserved in L. acidophilus species, which was generally versatile in growth on these compounds as compared to lactobacilli from other ecological niches or selected gut commensals from the Bacteroides, Bifidobacterium and Roseburia genera. The present disclosure therefore provides a surprising and unexpected aspect of carbohydrate metabolism in the human gut and highlights an important role of probiotic bacteria such as L. acidophilus, prevalent in the small intestine, in the bioconversion of distinct phytochemicals that exert beneficial effects on human health via absorption by the human host, or by altering microbiota composition.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of and consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

“Bioactivity” or “bioactive” as used herein relates to the effects a given substance exerts on a living system, cell, or organism. Generally, bioactivity of a substance involves the uptake of the substance into a living system, cell, or organism, such that the substance can exert a physiological effect on that living system, cell, or organism. In some cases, a cell or organism can interact with a substance to increase the bioactivity of that substance in another cell or organism (e.g., symbiosis). Increases in bioactivity often correlate with increases in

“Bioavailability” or “bioavailable” as used herein relates to the degree and/or rate at which a substance (e.g., phytochemical) is absorbed into a living system, cell or organism, or is made available at a site of physiological activity. The term “bioavailability” as used herein can indicate the fraction of an orally administered dose that reaches the systemic circulation as an intact substance, taking into account both absorption and local metabolic degradation. As would be recognized by one of skill in the art based on the present disclosure, there are many factors that influence bioavailability of a substance, including but not limited to, the degree to which a substance is or is not glycosylated. In some cases, bioavailability is associated with cell permeability, such that increases in cell permeability lead to increases in bioavailability. Generally, increases in bioavailability of a substance lead to uptake and metabolic utilization of that substance by a cell or organism, and may also facilitate the bioactivity of the substance. In some cases, a cell or organism can interact with a substance to increase the bioavailability of that substance in another cell or organism (e.g., symbiosis)

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some embodiments, the subject may be a human or a non-human. The subject or patient may be undergoing other forms of treatment.

“Variant” used herein with respect to a nucleic acid means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto,

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes may be identified, in part, by considering the hydropathic index of amino acids, as understood in the art. The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. Compositions

The present invention is directed to the field of microbiota research and therapy. In particular, the present disclosure provides compositions and methods for increasing bioavailability of phytochemicals using probiotic bacteria. Compositions and methods described herein include combinations of probiotic bacteria and prebiotic plant glycosides, wherein the probiotic bacteria are capable of converting the prebiotic plant glycosides into aglycones with increased bioavailability.

The small intestine is the primary site for absorption of nutrients and xenobiotics, which lends extra gravity to the metabolic activities of HGM prevalent in this part of the gastrointestinal tract, where probiotic bacteria, such as lactobacilli, constitute an important part of the microbial population. The present disclosure provides insight into the versatility of the probiotic bacterium, such as L. acidophilus NCFM, in utilization of dietary therapeutically active PGs, revealing that only the carbohydrate moieties are catabolized, while the aglycones are externalized making them bio-accessible to absorption by the host or further interactions with other organisms of the HGM.

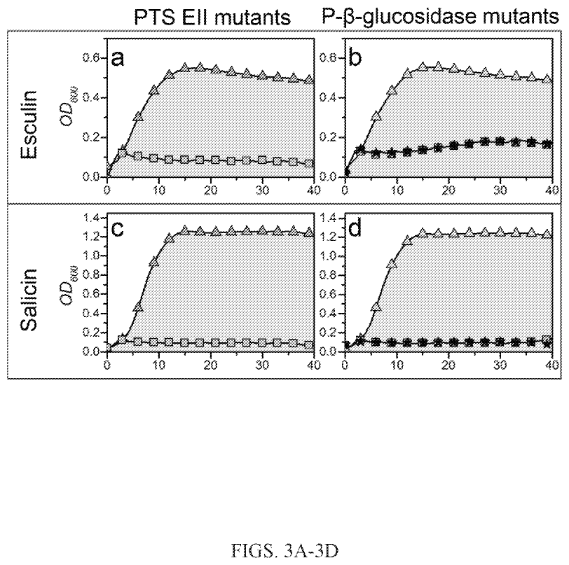

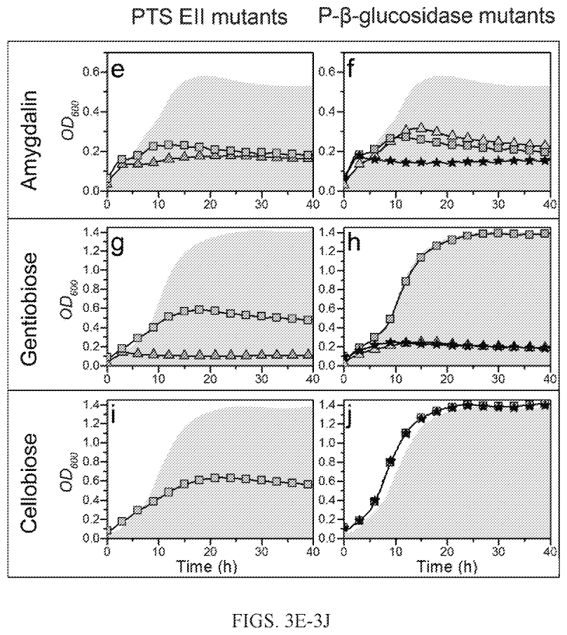

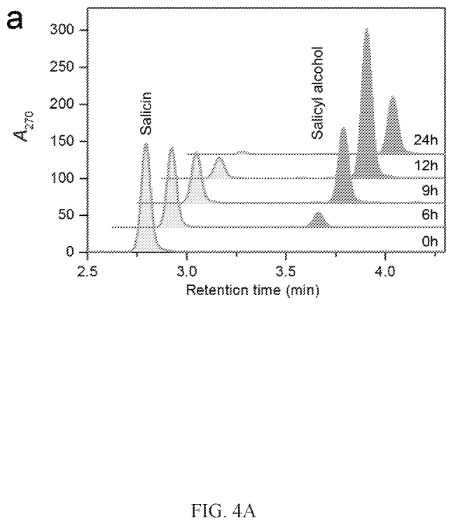

Carbohydrates are mainly taken up by PTS transporters in lactobacilli. Translocation is coupled to phosphorylation of the glycoside mostly at the 6′-position via an enzymatic cascade that relays the phosphoryl group to a substrate-specific enzyme II (EII) complex. The EIIC forms the translocation channel that defines the specificity of the EII complex. Phosphorylation is relayed via EIIA and BIB enzymes, of which the latter is known to interact specifically with EIIC. The EII modules are either encoded by a single gene (e.g., the EIICBA salicin and esculin uptake system (LBA0725)) or by separate genes to assemble the phosphorylation cascade. For example, the amygdalin EIIC component (LBA0227) requires coupling from EIIA and EIIB modules that are not encoded by the same locus. This EIIC is up-regulated upon growth on its substrate amygdalin, whereas the LBA0725 EIICBA is highly up-regulated on the substrates salicin and esculin, as well as on amygdalin (Table 6; HG. 3; and FIG. 5). Accordingly, inactivation of the EIIC elicits an impaired growth phenotype only on the substrate amygdalin, whereas the inactivation of the EIICBA causes about 50% reduction of growth on amygdalin as well as the two disaccharides cellobiose and gentiobiose, both not hydrolyzed by the LBA0225 P-Bgl encoded by this locus (FIG. 3).

The lack of growth on Amygdalin or gentiobiose, when the EIIC system is inactivated, precludes uptake of these compounds solely via the EIICBA system. A possible rationale for the co-regulation of the two transporters and the phenotypic impact of the EIICBA on non-substrates is that the EIIA and/or EIIB components of LBA0725 contribute in coupling phosphorylation to the amygdalin BIC system and possibly to other EIIC modules. The less drastic phenotype of the EIICBA on non-substrates, however, suggests the contribution of this transporter can be complemented by other PTS systems. As is currently known, this functional overlap between PTS systems that are assigned into different families has not been reported prior to the present disclosure. Such an overlap may orchestrate interplay between different transporters to confer the uptake of diverse sugars by bacteria.

Data from the present disclosure suggest a significant role of human gut probiotic bacteria, such as L. acidophilus, in the activation of dietary-relevant PGs (FIG. 5). For example, salicin is a pharmacologically inactive precursor of the analgesic and anti-rheumatic drug salicylic acid. Indeed salicylic acid has been the main metabolite (86%) in serum after oral administration of salicin-rich willow bark extract in humans. However, the present disclosure reveals that probiotic bacteria, such as L. acidophilus, performs a step in this bio-activation, via de-glycosylation and externalization of salicyl alcohol, which becomes accessible for oxidation to salicylic acid by other microbiota. Fraxin, which also sustains the growth of L. acidophilus, is one of the active ingredients in Chinese and Japanese herbal medicine and has several potential positive health effects including protection against oxidative stress. L. acidophilus also converts polydatin, which is enriched in wine and tea, to resveratrol that is one of the most studied therapeutic phytochemicals due to its implication in protection against e.g. inflammation, cancer, and obesity. Other lactobacilli have also been implicated in the metabolism of other PGs (e.g., the in vitro conversion of the isoflavonic daidzin present in soy products by Lactobacillus mucosae EPI2 to the estrogen-mimicking aglycone equol, which is proposed to be protective against breast cancer).

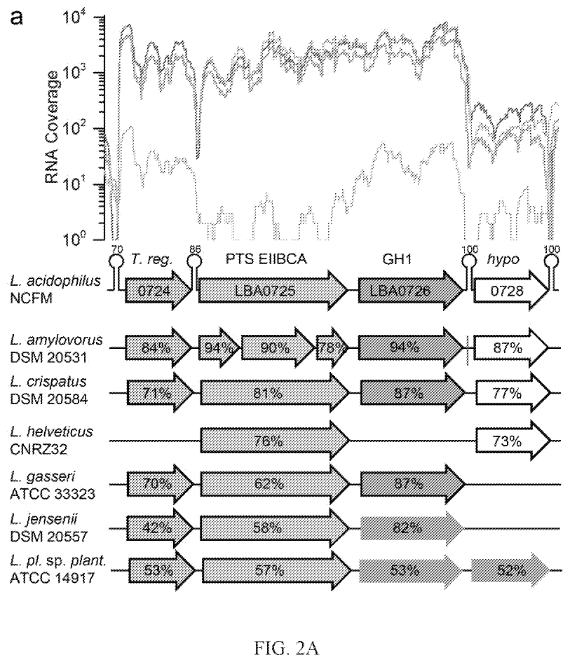

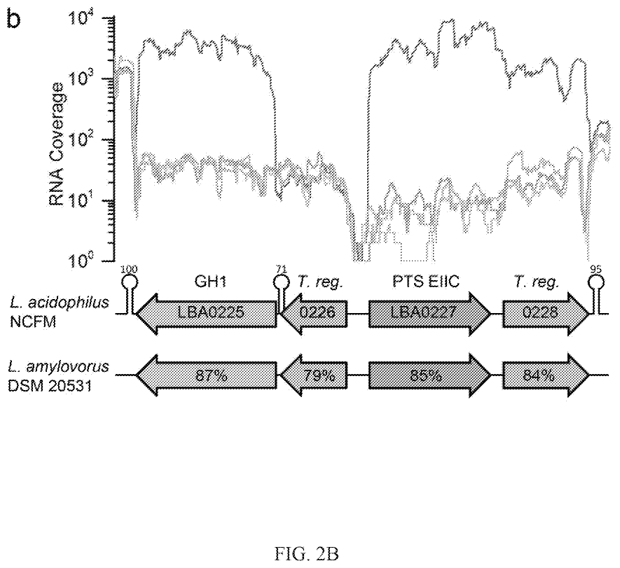

In siiico analysis of genomic sequences of L. acidophilus strains revealed the conservation of the PG utilization loci identified in the present disclosure, indicative of the potential ability of this species to metabolize PGs (Table 7). As described herein, growth analyses using four different PGs revealed large species variations in growth (Table 3), Generally, L. acidophilus were amongst the top strains in growth on PGs, and lactobacilli strains from the gut appeared to better at PGs utilization, compared to counterparts from other ecological niches, suggesting a competitive advantage in the adaptation to the human gut environment. Gene landscape analyses showed a correlation between growth on salicin and esculin and the presence of the intact LB 0724-6 locus in the tested strains that belong to the taxonomically closely related L. delbrueckii clade, i.e. L. acidophilus, Lactobacillus crispatus, Lactobacillus jensenii and Lactobacillus gasseri (FIG. 2A). Strains missing one or more genes within this cluster or having a fragmented version of the LBA0725 transporter gene, were conversely unable to grow on esculin and salicin (Table 3).

Taken together, the present disclosure provides surprising and unpredictable data regarding the bioconversion of PGs and the externalization of their bioactive aglycones by the human gut-adapted L. acidophilus and closely related taxa. The bioconversion of PGs is accompanied by a modulation of the activities of the phytochemicals in the small intestine, which renders these compounds bioavailable for further functional interplay with the host and other HGM taxa (FIG. 5). The present disclosure provides insight into the metabolism of plant derived glycosides and their bioconversion by microbiota with significant impact on human health.

a. Probiotic Bacterial Strains

Embodiments of the present disclosure include compositions having various types of probiotic bacterial strains, such as strains from the genus Lactobacillus. Probiotic bacterial strains of the present disclosure generally have the capability of converting a phytochemical (e.g., a plant phytochemical or a prebiotic plant glycoside) into a bioactive aglycone, or a derivative thereof. In some cases, probiotic bacterial strains of the present disclosure convert phytochemicals into aglycones through a deglycosylation mechanism involving one or more genes associated with the phosphotransferase system (PTS) or one or more genes that regulate intracellular hydrolysis of plant glycosides. Generally, an aglycone is an organic compound that remains after a glycosyl group on a glycoside is replaced with a hydroxyl group. Removal of the glycosyl group from a phytochemical or a plant glycoside can increase the bioavailability or bioactivity of the aglycone, as described in the data below. Probiotic bacterial strains capable of internalizing and/or absorbing phytochemicals and releasing bioavailable and bioactive aglycones include, but are not limited to, L. acidophilus, L. amylovorus, L. animalis, L. crispatus, L. Jermentuni, L. gasseri, L. helveticus, L. intestinalis, L. jensenii, L. johnsonii, L. plantarum, L. reuteri, L. rhamnosus, and combinations thereof. In some embodiments, probiotic bacterial strains that can be used in the compositions of the present disclosure include one more of L. acidophilus LA-1, L. acidophilus NCFM, L. amylovorus (ATCC 33620, DSM 20531), L. animalis (DSM 20602), L. crispatus (ATCC 33820, DSM 20:584), L. jermentum (ATCC 14931), L. gasseri (ATCC 33323), L. helveticus CNRZ32, L. intestinalis Th4 (ATCC 49335, DSM 6629), L. jensenii (ATCC 25258, 62G-, DSM 20557), L. johnsonii (ATCC 33200), L. plantarum sp. plantarum (ATCC 14917, LA.70), L. reuteri (ATCC 23272, DSM 20016), L. rhamnosus GG (ATCC 53103), or combinations thereof.

Embodiments of the present disclosure can also include compositions having various types of probiotic bacterial strains, in addition to, and distinct from, the probiotic Lactobacillus strains mentioned above. For example, compositions of the present disclosure can include a probiotic bacterial strain capable of converting a prebiotic plant glycoside into a bioactive aglycone, or derivative thereof, as well as an additional probiotic bacterial strain, including but not limited to, a bacterial strain from the genus Bifidobacterium, Roseburia, Weissella, Enterococcus, Lactococcus, Eubacterium, Butirivibrio, Clostridium group XIVa, or combinations thereof, and in some cases, Bacteroides. Other probiotic bacterial strains can also be included, as would be recognized by one of ordinary skill in the art based on the present disclosure.

Probiotic bacterial strains of the present disclosure can convert phytochemicals into aglycones through a deglycosylation mechanism involving one or more genes associated with the phosphotransferase system (PTS), or one or more genes that regulate intracellular hydrolysis of plant glycosides. One or more genes associated with the PTS system include, but are not limited to, a LicT transcriptional anti-terminator, an EIICBA component of the PTS system, a phospho-3-glucosidase of glycoside hydrolase family 1 (GH1), or any homologous glycosidases and hydrolases. One or more genes associated with the regulation or modulation of the intracellular hydrolysis of plant glycosides include various enzymes that hydrolyze or phosphorylate a plant glycoside, such as any member of the GH1 to GH128 families of glycoside hydrolases, for example, a member of GH1, GH2, GH3, and GH94, and other members of different glycoside hydrolase families, such as GH78 putative α-L-rhamnosidase. In some embodiments, the gene is a plant glycoside hydrolase, such as one or more phospho-β-glucosidases (P-Bgls), β-glucosidases, or rhamnosidases.

Additionally, probiotic bacterial strains of the present disclosure can convert phytochemicals into aglycones through a deglycosylation mechanism involving a genetic alteration in one or more genes associated with the phosphotransferase system (PTS), or a genetic alteration in one or more genes that regulate intracellular hydrolysis of plant glycosides. As would be readily apparent to one of ordinary skill in the art based on the present disclosure, genetic alterations in any of the aforementioned genes or genetic loci can be accomplished by conventional means known in the art. Depending on the desired functional outcome, any of these genes or genetic loci can be altered to create loss-of-function alleles, gain-of-function alleles, hypermorphs, hypomorphs, and the like. Generally, a genetic alteration includes any change from the wild-type or reference sequence of one or more nucleic acid molecules. Genetic alterations include without limitation, base pair substitutions, additions and deletions of at least one nucleotide from a nucleic acid molecule of known sequence.

b. Prebiotic Plant Glycosides

Embodiments of the present disclosure include compositions having various types of phytochemicals, such as prebiotic plant glycosides, capable of being converted to aglycones. In some embodiments, prebiotic plant glycosides include, but are not limited to, an aromatic glycoside, including but not limited to a coumarin glucoside, a stilbenoid glucoside, an aryl β-D-glucoside, a resveratrol glucoside derivative, a flavonol, a phenolic, a polyphenolic or combinations thereof. In some embodiments, prebiotic plant glycosides include, but are not limited to, a glucoside, a fructoside, a rhamnoside, a xyloside, an arabinopyranoside, a glucuronide, or combinations thereof. In other embodiments, prebiotic plant glycosides include, but are not limited to, a mono- or di-glucoside anomerically substituted with a single or double aromatic ring system. In still other embodiments, prebiotic plant glycosides include, but are not limited to one or more of Amygdalin, Arbutin, Aucubin, Daidzin, Esculin, Fraxin, Isoquercetin, Polydatin, Rutin hydrate, Salicin, Sinigrin hydrate, Vanilin 4-O-β-glucoside, or glucoside derivatives thereof. Other prebiotic plant glycosides can also be included, as would be recognized by one of ordinary skill in the art based on the present disclosure.

c, Carriers and Excipients

Embodiments of the present disclosure can also include compositions having various physiologically acceptable carriers and/or excipients. For example, physiological carriers or excipients can include various substances that facilitate the formation, digestion, and/or metabolism of a composition that includes a probiotic bacterial strain and a prebiotic plant glycoside. Physiologically acceptable excipients and carriers can include, but are not limited to, one or more of cellulose, microcrystalline cellulose, mannitol, glucose, sucrose, trehalose, xylose, skim milk, milk powder, polyvinylpyrrolidone, tragacanth, acacia, starch, alginic acid, gelatin, dibasic calcium phosphate, stearic acid, croscarmellose, silica, polyethylene glycol, hemicellulose, pectin, amylose, amylopectin, xylan, arabinogalactan, polyvinylpyrrolidone, and combinations thereof. In sonic embodiments, a. probiotic bacterial strain and a prebiotic plant glycoside can be combined with various nontoxic, physiologically acceptable carriers for tablets, pellets, capsules, troches, lozenges, aqueous or oily suspensions, dispersible powders or granules, suppositories, solutions, emulsions, suspensions, hard or soft capsules, caplets or syrups or elixirs and any other form suitable for use. Carriers can include, lactose, gum acacia, gelatin, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents can also be used.

### 3. Nutritional Supplement

Embodiments of the present disclosure also provide compositions that include a probiotic bacterial strain, a prebiotic plant glycoside, and a physiologically acceptable carrier and/or excipient that can be formulated as a nutritional supplement or nutraceutical and wherein the probiotic bacterial strain is capable of converting the prebiotic plant glycoside into a bioactive aglycone, or derivative thereof. Any of the aforementioned components can be used to formulate such a nutritional supplement, such that it can be administered to a subject. A nutritional supplement containing a probiotic bacterial strain, a prebiotic plant glycoside, and a physiologically acceptable carrier and/or excipient can be formulated and administered in various forms, including but not limited to, a tablet, pill, capsule, powder, lozenge, or suppository. In some embodiments, a nutritional supplement containing a probiotic bacterial strain, a prebiotic plant glycoside, and a physiologically acceptable carrier and/or excipient can be formulated with a fermentable dairy product, such as yogurt, cheese, cream cheese, cottage cheese, and the like.

The compositions according to the present disclosure can be formulated according to the mode of administration to be used. For example, in cases where the compositions are injectable compositions, they can be formulated as sterile, pyrogen free and particulate free compositions. Additives for isotonicity can also be used and include sodium chloride, dextrose, mannitol, sorbitol and lactose. In some cases, isotonic solutions such as phosphate buffered saline are advantageous. Stabilizers can include gelatin and albumin. In some embodiments, a vasoconstriction agent is added to the formulation.

The composition may further comprise a pharmaceutically acceptable excipient. The pharmaceutically acceptable excipient may be functional molecules as vehicles, adjuvants, carriers, or diluents. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

### 4. Methods of Use

Embodiments of the present disclosure also provide methods for using the compositions and nutritional supplements, as described above. The present disclosure is directed to methods for providing a dietary supplement to a subject by administering to the subject a composition containing a probiotic bacterial strain, a prebiotic plant glycoside, and a physiologically acceptable carrier and/or excipient, as described above, or nutritional supplement thereof, wherein the probiotic bacterial strain is capable of converting the prebiotic plant glycoside into a bioactive aglycone, or derivative thereof. The present disclosure is also directed to methods of supplementing a fermented dairy product by mixing a composition containing a probiotic bacterial strain, a prebiotic plant glycoside, and a physiologically acceptable carrier and/or excipient, as described above, or nutritional supplement thereof, with a fermented dairy product, wherein the probiotic bacterial strain is capable of converting the prebiotic plant glycoside into a bioactive aglycone, or derivative thereof.

### 5. Methods of Treating a Condition in a Subject

Embodiments of the present disclosure also provide methods of treating one or more conditions in a subject with a composition containing a probiotic bacterial strain, a prebiotic plant glycoside, and a physiologically acceptable carrier and/or excipient, wherein the probiotic bacterial strain is capable of converting the prebiotic plant glycoside into a bioactive aglycone, or derivative thereof. Any of the above nutritional supplement formulations can be used to treat one or more conditions in a subject. For example, compositions of the present disclosure can be used to treat a disorder or disease associated with a deficiency in one or more phytochemicals, or the bacterial strains able to render them bioavailable. In some cases, compositions of the present disclosure can be used to treat a disease or disorder that exists (or has an etiology) independent of the presence or absence of one or more phytochemicals, or the bacterial strains able to render them bioavailable. In such cases, increasing the bioavailability or bioactivity of the one or more phytochemicals using the compositions of the present disclosure can cure, alleviate, modulate, treat, and/or prevent the disease or disorder. In other cases, compositions of the present disclosure can be used to treat a disease or disorder that is not currently known to be associated with a deficiency in a particular phytochemical.

Generally, administering to a subject in need of treatment a composition containing a probiotic bacterial strain, a prebiotic plant glycoside, and a physiologically acceptable carrier and/or excipient can lead to an increase in the bioavailability and/or bioactivity of the prebiotic plant glycoside (e.g., deglycosylation), which treats a disease or disorder. Without being limited to a particular mechanism, treating a disease or disorder can involve a probiotic bacterial strain (e.g., documented probiotic strains from various species within the Lactobacillus genus) coming into contact with and internalizing a prebiotic plant glycoside. The probiotic bacterial strain can then convert the plant glycoside into a bioactive aglycone, or an aglycone derivative. In some cases, after conversion of the plant glycoside into a bioactive aglycone, the probiotic bacteria can release the aglycone, such that it is bioavailable to a host subject or other microbiota taxa.

Conditions that can be treated in this manner include, but are not limited to, one or more of obesity, cardiovascular disease, metabolic syndrome, cancer, autoimmune disease, inflammatory disorder, digestive system disorder, digestive system-related disorder, or combinations thereof. Other diseases and disorders knowns to be affected by a prebiotic plant glycoside, an aglycone, or derivatives thereof, are also contemplated, as would be recognized by one of ordinary skill in the art based on the present disclosure.

Embodiments of compositions containing a probiotic bacterial strain, a prebiotic plant glycoside, and a physiologically acceptable carrier and/or excipient can be formulated and administered in various forms and in various dosages. In some embodiments, compositions can be formulated to contain a dosage of probiotic bacteria ranging from about 1 mg to about 100 mg. In some embodiments, compositions can be formulated to contain a dosage of probiotic bacteria ranging from about ling to about 50 mg, from about 1 mg to about 40 mg, from about 1 mg to about 30 mg, from about 1 mg to about 20 mg, or from about 1 mg to about 10 mg. In some embodiments, compositions can be formulated to contain a dosage of probiotic bacteria ranging from about 10 mg to about 100 mg, from about 20 mg to about 100 mg, from about 30 mg to about 100 mg, from about 40 mg to about 100 mg, or from about 50 mg to about 100 mg.

Additionally, compositions can be formulated to contain a dosage of a prebiotic plant glycoside ranging from about ling to about 500 mg. In some embodiments, compositions can be formulated to contain a dosage of a prebiotic plant glycoside ranging from about 1 mg to about 50 mg, from about 1 mg to about 40 mg, from about 1 mg to about 30 mg, from about 1 mg to about 20 mg, or from about 1 mg to about 10 mg. In some embodiments, compositions can be formulated to contain a dosage of a prebiotic plant glycoside ranging from about 10 mg to about 100 mg, from about 20 mg to about 100 mg, from about 30 mg to about 100 mg, from about 40 mg to about 100 mg, from about 50 mg to about 100 mg, from about 60 mg to about 100 mg, from about 70 mg to about 100 mg, from about 80 mg to about 100 mg, or from about 90 mg to about 100 mg in some embodiments, compositions can be formulated to contain a dosage of a prebiotic plant glycoside ranging from about 100 mg to about 500 mg, from about 150 mg to about 500 mg, from about 200 mg to about 500 mg, from about 300 mg to about 100 mg, from about 350 mg to about 500 mg, from about 400 mg to about 100 mg, or from about 450 mg to about 500 mg.

Dosing regimens may vary, depending on the needs of the subject, the type of condition, the dosing regimen, and other treatment variables that would be recognized by one of ordinary skill in the art. For example, dosing may include a daily dose, such that the compositions are formulated to be administered once per day. Dosing regimens and formulations can also include administration of the compositions of the present disclosure multiple times per day, weekly, hi-weekly, and monthly.

### 6. Examples

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present invention has multiple aspects, illustrated by the following non-limiting examples.

### EXAMPLE 1

### Materials and Methods

Chemicals and Carbohydrates. The plant glycosides utilized in the present disclosure are described below Table 1. All other chemicals used were of high purity.

TABLE 1
Plant glycosides and their ability to support growth (OD₆₀₀) of L. acidophilus NCFM.

| | | Example of | | | | | |
|---|---|---|---|---|---|---|---|
| | | common | | | Growth | | Catabolism |
| | | natural | | | concentration | OD₆₀₀ | based on |
| Compound | CAS No. | source | Supplier | Purity^{a} | (w/v)^{b} | max^{c} | MS^{d} |
| Amygdalin | 29883-15-6 | Almonds | Sigma | ≥99% | 1% | 0.3 | Yes |
| Arbutin | 497-76-7 | Pear | Sigma | ≥98% | 1% | 0.0 | No |
| Aucubin | 479-98-1 | Asterid | Chemfaces | ≥98% | 0.5% | 0.0 | No |
| | | plants | | | | | |
| Daidzin | 552-66-9 | Soy | AdooQ | ≥98% | 0.5% | 0.0 | ND |
| Esculin | 531-75-9 | Dandelion | Sigma | ≥98% | 0.5% | 0.4 | Yes |
| | | coffee | | | | | |
| Fraxin | 524-30-1 | Kiwi | Chemfaces | ≥98% | 0.5% | 0.4 | Yes |
| Isoquercetin | 482-35-9 | Onion | Sigma | ≥90% | 0.5% | 0.0 | No |
| Polydatin | 65914-17-2 | Grapes | Sigma | ≥95% | 0.5% | NA | Yes |
| Rutin | 153-18-4 | Tea | Sigma | ≥94% | 0.5% | 0.1 | No |
| hydrate | | | | | | | |
| Salicin | 138-52-3 | Willow tree | Sigma | ≥99% | 1% | 0.8 | Yes |
| Sinigrin | 3952-98-5 | Broccoli | Sigma | ≥99% | 0.5% | 0.0 | ND |
| hydrate | | | | | | | |
| Vanilin 4-O- | 494-08-6 | Vanilla | mybiosource.com | 100% | 0.5% | 1.3 | Yes |
| β-glucoside | | | | | | | |

^{a}As provided by supplier.
^{b}Concentration in single carbon source growth experiments.
^{c}Maximum optical density (600 nm) corrected for growth in semi-defined medium without carbon source in 200 μl cultures in 96 microtitre plates, which corresponds to approximately 50% of the absorbance in a 1 cm cuvette.
^{d}Mass spectrometry qualitative analysis of catabolism based on the depletion of the plant glycoside and/or appearance of its metabolites.
ND: not detected.
NA: Not applicable due to low solubility of the compound.

Bacterial Strains and Growth. Bacterial strains and plasmids are presented in Table 2. below.

TABLE 2
Strains used and constructed for gene deletion mutants in L. acidophilus NCFM.

| Strain | Source, genotype or characteristics/description |
|---|---|
| Escherichia coli EC101 | RepA⁺ JM101; Km^{r}; repA gene from integration of pWV01 in |
| | the chromosome; cloning host for pORI-based plasmids+ |
| Lactobacillus acidophilus | |
| NCFM | Human intestinal isolate |
| NCK1909 (Δupp) | NCFM with a 0.3 kb in-frame deletion within the upp gene |
| | (LBA0770); background/parent strain for NCFM deletion |
| | mutants |
| NCK1910 | NCK1909 harboring the plasmid pTRK669 (17) |
| NCK2416 (ΔLBA0225) | NCK1909 with a 1.3 kb in-frame deletion within LBA0225 |
| NCK2418 (ΔLBA0227) | NCK1909 with a 1.2 kb in-frame deletion within LBA0227 |
| NCK2422 (ΔLBA0725) | NCK1909 with a 1.9 kb in-frame deletion within LBA0725 |
| NCK2424 (ΔLBA0726) | NCK1909 with a 1.3 kb in-frame deletion within LBA0726 |
| NCK2426 | NCK2416 with a 1.3 kb in-frame deletion within LBA0726 |
| (ΔLBA0225ΔLBA0726) | |

Lactobacillus strains were propagated statically in de Man-Rogosa-Sharpe (MRS) broth (Difco Laboratories, Detroit, Mich., USA) under aerobic conditions or on MRS agar plates (1.5% (w/v), Difco) under anaerobic conditions at 37° C. or at 42° C. for pTRK669 elimination. Recombinant L. acidophilus strains were selected in the presence of 2 μg mL⁻¹ erythromycin (Sigma-Aldrich, St. Louis, Mo., USA) and/or 2-5 μg mL ⁻¹ chloramphenicol (Sigma). Selection of plasmid-free double recombinants was done on a semi-defined agar medium containing 2% (w/v) glucose (GSDM) and 100 μg mL ⁻¹ 5-fluorouracil (5-FU) (Sigma) as described by Goh et al.

For initial growth and gene expression studies, L. acidophilus NCFM was propagated three times in semi-defined medium (SDM) supplemented with either 1% or 0,5% (w/v) of the plant glycoside or carbohydrate (Table 1). For the RNA-seq analysis, cells were harvested by centrifugation (3,220×10 min, 25° C.) in the mid-exponential phase (OD₆₀₀=0.6-0.8) and stored at −80° C. for subsequent RNA isolation. For the mass spectrometry metabolite analyses, 200 μt samples were taken at 0, 3, 6, 9, 12, and 24 hours of growth; cells were removed by centrifugation; and supernatants were stored at −80° C. for further analysis.

Phenotypic growth assays were performed using 1% (v/v) overnight cultures of L. acidophilus strains (Table 2) and other Lactobacillus species (Table 3) grown on SDM supplemented with 1% (w/v) glucose to inoculate 200 μL of SDM supplemented with 1% (w/v) of the examined carbohydrate (0.5% in the case of esculin) in 96-well microplate wells (Corning Costar, Corning, N.Y., USA) in duplicate or triplicate wells, respectively. The microplates were sealed with clear adhesive film, incubated at 37° C. in a Fluostar Optima microplate reader (BMG Labtech, Cary, N.C., USA), and the cell optical density (OD₆₀₀) was monitored for 30 hours.

TABLE 3
Growth of Lactobacillus species on selected plant glucosides, cellobiose, and glucose corrected to the
growth level in medium without carbohydrate.

| | | Genome | | | | | | |
|---|---|---|---|---|---|---|---|---|
| Strain | Source (ref) | sequence | Amygdalin | Arbutin | Esculin | Salicin | Cellobiose | Glucose |
| L. acidophilus LA-1 | Human | | ++ | − | +++ | +++ | +++ | +++ |
| L. acidophilus NCFM | Human intestinal | Complete | + | − | ++ | +++ | ++ | ++ |
| | isolate | | | | | | | |
| L. amyloverus ATCC | Cattle feces | | − | − | − | − | +++ | +++ |
| 33620, DSM 20531 | | | | | | | | |
| L. animalis DSM 20602 | Baboon dental plaque | | − | + | + | + | + | + |
| L. crispatus ATCC 33820, | Human isolate | | − | − | ++ | +++ | − | ++ |
| DSM 20584 | | | | | | | | |
| L. fermentum ATCC 14931 | Fermented beets | | − | − | − | − | − | +++ |
| L. gasseri ATCC 33323 | Human isolate | | − | − | ++ | ++ | ++ | +++ |
| L. helveticus CNRZ32 | Industrial cheese | | + | − | − | − | + | ++ |
| | starter culture | | | | | | | |
| L. intestinalis Th4, ATCC | Rat intestine | Scaffold | − | − | − | − | − | ++ |
| 49335, DSM 6629 | | | | | | | | |
| L. jensenii ATCC 25258, | Human vaginal | Scaffold | + | + | ++ | ++ | ++ | ++ |
| 62G, DSM 20557 | discharge | | | | | | | |
| L. johnsonii ATCC 33200 | Human blood | Contig | − | + | − | + | + | +++ |
| L. plantarum sp. plantarum | Pickled cabbage, | Scaffold | +++ | +++ | ++ | +++ | +++ | +++ |
| ATCC 14917, LA70 | human microbiome | | | | | | | |
| | project | | | | | | | |
| L. reuteri (ATCC 23272, | Human feces | | − | − | − | − | − | ++ |
| DSM 20016) | | | | | | | | |
| L. rhanmosus GG (ATCC | Human feces | | +++ | +++ | ++ | +++ | +++ | +++ |
| 53103) | | | | | | | | |

“+++” signifies OD600 max > 0.6.
“++” signifies 0.6 > OD₆₀₀ max > 0.3.
“+” signifies 0.3 > OD₆₀₀ max > 0.1.
“−” signifies OD₆₀₀ max < 0.1.

Escherichia coli EC101 used for generating the L. acidophilus gene knock-outs was grown in Brain Heart Infusion (BHI) broth (Difco) at 37° C. with aeration in the presence of kanamycin (40 μg mL⁻¹). Recombinant E. coli EC101 containing pTRK935-based plasmids were selected with erythromycin (150 μg mL⁻¹). Growth of Bifidobacterium longum sp. longum DSM 20219, Bifidobacterium longum sp. infantis DSM 20088 and Bacteroides ovatus DSM 1896 was carried out in MRS medium or modified MRS medium supplemented with a 1% (w/v) carbon source. Roseburia intestinalis L1-82 was cultured in YCFA medium supplement with a carbon source.

RNA Extraction, Sequencing and Transcriptional Analysis. Pellets from 10 mL cell cultures were resuspended in 1 mL of TRI Reagent (Thermo Fisher Scientific, Waltham, Mass.) and thereafter transferred into 1.5 mL bead-beating conical tubes with 0.1 mm glass beads (BioSpec Products, Inc., Bartlesville, Okla., USA), and cells were disrupted by 6×1 min cycles (with 1 min on ice intermittently) with a Mini-Beadbeater-16 (BioSpec Products). RNA purification was performed using the Direct-zol RNA MiniPrep kit (Zymo Research, Irvine, C USA) with on-column DNase I treatment followed by an additional Turbo DNAse (Thermo Fisher) treatment of the eluted RNA, and further purification was carried out using the RNA Clean & Concentrator-5 kit (Zymo Research). The quality of RNA was analyzed using an Agilent 2100 Bioanalyzer (Agilent Technologies, Santa Clara, Calif., USA) and the absence of genomic DNA was confirmed by PCR using L. acidophilus NCFM gene-specific primers. Library preparation and RNA sequencing was performed by the High-Throughput Sequencing and Genotyping Unit of the Roy J. Carver Biotechnology Center, University of Illinois (Urbana-Champaign, Ill., USA). After rRNA removal (Ribo-Zero rRNA Removal Kit, Bacteria, Illumina, San Diego, Calif., USA), library preparation was carried out using the TruSeq Stranded Total RNA Library Prep kit (Illumina). Single-read RNA sequencing was performed using a HiSeq 2500 Ultra-High-Throughput Sequencing System (Illumina) and the Illumina HiSeq SBS Kit v4 (Illumina) with a read length of 160 nt. The raw reads were de-multiplexed with the bcl2fastq Conversion Software (v2.17.1.14, Illumina); trimmed for the adaptor sequences, quality trimmed to remove sequence reads with an error probability threshold of 0.001 (Phred score, 30) and filtered to remove reads <20 nt using Geneious version 9.0.460. The quality of the reads was assessed by FastQC v0.11.5 (www.bioinformatics.babraham.ac.uk/projects/fastqc/). The resulting reads were then mapped to the L. acidophilus NCFM reference genome using the Geneious Mapper with default settings. The sequencing coverage depths were calculated to be 610-692x, and transcriptional analyses were based on normalized transcripts per million (nTPM) as calculated within Geneious. Differentially expressed genes were defined as having a log₂ ratio≥2 unless otherwise stated.

RT-qPCR Assay. To confirm the results of the RNA-seq transcriptional study, reverse transcriptase quantitative PCR (RT-qPCR) analysis of selected genes was performed. Briefly, the iTaq Universal SYBR Green One-Step Kit (Bio-Rad Laboratories, Hercules, Calif., USA) was used according to manufacturer's instructions, except for scaling down to 25 μL reactions with 50 ng of RNA template—and 300 nM of each primer (Table 4). An iCycler MyiQ single color detection system (Bio-Rad) was used and the data were analyzed using iCycler MyiQ software v1.0 (Bio-Rad). The correlation coefficients for the standard curves and PCR efficiencies were between 0.930-0.999, and 88.7-102.5%, respectively.

TABLE 4
Primers

| | | Primer | SEQ |
|---|---|---|---|
| Name | Sequence | usage | ID NO: |
| LBA0225A | GTTAATAGGATCCCAACCATAGTTCATATCAAGTGGAA | PCR | 1 |
| LBA0225B | AAGTTGATGAGCGGCAACAG | PCR | 2 |
| LBA0225C | CTGTTGCCGCTCATCAACTTCAAAATGTGATTAAAAC | PCR | 3 |
| | AAATGGCC | | |
| LBA0225D | TTAGTAGAGCTCGACTTGCATGCACCACAAAT | PCR | 4 |
| LBA0225up | TGCTCAAAACGCACATGTTTCA | Seq/Control | 5 |
| LBA0225down | ACTCGTGCTCGTGAACCAAT | Seq/Control | 6 |
| LBA0225mid | GAACACTATGTTCCATCTTAGGAAAA | Seq/Control | 7 |
| LBA0227A | GTAATAGGATCCGGTAGTATTAGCTAATTTAGGAACA | PCR | 8 |
| LBA0227B | TAATGCAACGATTGGTCTTG | PCR | 9 |
| LBA0227C | CAAGACCAATCGTTGCATTACTCTACAAGCAGGAACA | PCR | 10 |
| | ACA | | |
| LBA0227D | TTAGTAGAATTCAATCCTTATTTCCGGTAGCT | PCR | 11 |
| LBA0227up | GTTGTTAACGAATCTGTTGATCA | Seq/Control | 12 |
| LBA0227down | ATCGTTTAAAAATTGCCATTGC | Seq/Control | 13 |
| LBA0227mid | TCAACGGTAGATAATGACGA | Seq/Control | 14 |
| LBA0227.F | AGATGCAGAACACGGTGGTC | RT-qPCR | 15 |
| LBA0227.R | GTCCAATAGTCATTCCTGCACC | RT-qPCR | 16 |
| LBA0383.F | TACTCAAAGAAGGCTTACG′ | RT-qPCR | 17 |
| LBA0383.R | ATTAACTACGGCTTGAACC | RT-qPCR | 18 |
| LBA0574.F | GGCAACCGTTGTGATGGTIATC | RT-qPCR | 19 |
| LBA0574.R | ACCTTGCAAAGTTTCTTGGGC | RT-qPCR | 20 |
| LBA0606.F | TACCGGTCTTCACCACTTGG | RT-qPCR | 21 |
| LBA0606.R | GCTGCGTATTCTGCAAGGTG | RT-qPCR | 22 |
| LBA0725A | GTAATAGGATCCTCACATTGATTTTGCCGTTACT | PCR | 23 |
| LBA0725B | TCTTTGCCACCAACATCTTT | PCR | 24 |
| LBA0725C | AAAGATGTTGGTGGCAAAGAACATCAGTTAATGGAC | PCR | 25 |
| | AAGTGC | | |
| LBA0725D | TTAGTAGAGCTCTCTAGCATCATTACGGCTGT | PCR | 26 |
| LBA0725up | CAGGTTAAAGAGTTTAAATCACAAACA | Seq/Control | 27 |
| LBA0725down | CACGAGCACTTGCAACAAAT | Seq/Control | 28 |
| LBA0725mid | TGAACTGGACATTAGATTCAGACGA | Seq/Control | 29 |
| LBA0725.F | ATCTTCGGTGTTCACTGGGG | RT-qPCR | 30 |
| LBA0725.R | AAACAACCCCGATTTGTGCG | RT-qPCR | 31 |
| LBA0726A | GTAATAGGATCCAAGTCAGTAGATGCAAAATATGA | PCR | 32 |
| LBA0726B | GTAGGCACCTTCAATTTGAT | PCR | 33 |
| LBA0726C | ATCAAATTGAAGGTGCCTACTCACTTAAGAGACTTCC | PCR | 34 |
| | TAAGGA | | |
| LBA0726D | TTAGTAGAATTCAGTCCGCTTGTCATCATAGT | PCR | 35 |
| LBA0726up | AAGGGGGTTCAATGACTCAAA | Seq/Control | 36 |
| LBA0726down | GCTTCATACAAAAATTCAGATTTGACA | Seq/Control | 37 |
| LBA0726mid | TTGTTAAAGGTGAAGTAAAGGTAGG | Seq/Control | 38 |
| LBA1611.F | TGCTTGGTCCTTAGCTGGTG | RT-qPCR | 39 |
| LBA1611.R | CAATGCCGCAGTAACCGAAG | RT-qPCR | 40 |
| LBA1812.F | TCCCAGATACCTGAAACGCC | RT-qPCR | 41 |
| LBA1812.R | AAATGAAGTTTGGCCAGGCG | RT-qPCR | 42 |
| LBA1872.F | CCGCGTTGCAGATACATCAAC | RT-qPCR | 43 |
| LBA1872.R | TCACAACCCACGCTTTATTGG | RT-qPCR | 44 |

DNA Manipulation and Transformation. Genomic DNA from L. acidophilus NCFM and mutants thereof was isolated using the ZR Fungal/Bacterial DNA MiniPrep kit (Zymo Research). Plasmic' DNA was isolated using the QIAprep Spin MiniPrep kit (Qiagen, Hilden, Germany). Restriction enzymes were from Roche (Roche, Basel. Switzerland), and T4 DNA ligase was from NEB (New England Biolabs, Ipswich, Mass., USA). PfuUltra II fusion HS DNA polymerase (Agilent Technologies, Santa Clara, Calif., USA) was used for cloning and Choice-Taq Blue DNA polymerase (Denville Scientific. South Plainfield, N.J., USA) for PCR screening of recombinants. PCR amplicons were analyzed on 0.8% (w/V) agarose gels and extracted using the QIAquick Gel Extraction kit (Qiagen). DNA sequencing was performed by Eton Biosciences (Durham, N.C. USA).

Construction of gene deletion mutants. The L. acidophilus NCFM genes LBA0225 and LBA0726, both encoding P-Bgl of glycoside hydrolase family 1 (GH1) enzymes in addition to the LBA0227 and LBA0725 genes encoding an EIIC and an EIICBA components of two PTS systems, respectively, were deleted using the upp-based counterselectable gene replacement system. Briefly, in-frame deletions were constructed by amplifying 650-750 bp of the up- and downstream flanking regions of the deletion targets with two primer pairs (e.g., LBA0225A/LBA0225B and LBA0225C/LBA0225D; Table 4). The resulting purified products were joined by splicing using overlap extension PCR (SOE-PCR63) and amplified to establish the deletion alleles. The SOE-PCR products, that include flanking restriction enzyme sites, were cloned within the BamHI and SacI/EcoRI sites of the pTRK935 integration vector and transformed into E. coli EC101. The resulting recombinant plasmids (pTRK1113-6) were confirmed by DNA sequencing and electroporated into L. acidophilus NCK1910 (Table 2) that contains the pTRK669 helper plasmid, and the recovery of the single- and double-crossover recombinants was performed as previously described. Recombinants carrying the new gene deletion alleles were isolated by colony PCR using primer pairs denoted up/down (e.g., LBA0225up/LBA0225down), which anneal to the flanking regions of the amplicons. Sequence integrity and in-frame deletions were verified by DNA sequencing employing the aforementioned primer pairs and primer denoted mid (e.g., LBA0225mid). The mutations were in-frame deletions of 90-96% of the coding regions.

Analysis of plant glycoside uptake from L. acidophilus NCFM culture supernatants using mass spectrometry. The supernatants of L. acidophilus NCFM cultures grown on amygdalin, arbutin, esculin or salicin as carbon sources were analyzed during 24 hours by ultra-high performance liquid chromatography-diode array detection-quadruple time of flight mass spectrometry (UHPLC-DAD-Q-TOF-MS). Samples were diluted 1:20 (v/v) with methanol and an injection volume of 1.5 was used. Separation was carried out on an Agilent Poroshell 120 phenyl-hexyl column (2.1×150 mm, 2.7 μm) using the Agilent Infinity 1290 UHPLC system (Agilent Technologies, Santa Clara, Calif., USA) equipped with a UV/vis spectrum diode array detector. Separation was performed at 0.35 mL min-1., 60° C. with a linear gradient consisting of water (A) and acetonitrile (B) both buffered with 20 mM formic acid, starting at 10% B and increased to 100% in 15 min where it was held for 2 min, returned to 10% in 0.1 min and kept for 3 min. MS detection was performed on an Agilent 6550 iFunnel QTOF MS equipped with Agilent Dual Jet Stream electrospray ion source with the drying gas temperature of 160° C. and gas flow of 13 L min⁻¹, whereas the sheath gas temperature was 300° C. and flow was 16 L min-1. Ionization was conducted in ESI-mode with capillary voltage set to 4000 V and nozzle voltage to 500 V. Mass spectra were recorded as centroid data for m/z 85-1700 in MS mode with an acquisition rate of 10 spectra s⁻¹. To avoid carry-over, the needle seat was back-flushed for 15 s at 4 mL min-1 with each of: i) isopropanol: 0.2% ammonium hydroxide (w/v) in water (1:1 v/v); ii) acetonitrile with 2% formic acid (w/v); iii) water with 2% formic acid. Data was processed with the Agilent Mass Hunter Qualitative Analysis B.07.00 software package (Agilent Technologies) and molar concentrations were obtained from standard curves of the plant glycosides and their main metabolites. Targeted compound searches were performed using lists of previously identified compounds plus standard chemical modifications.

### EXAMPLE 2

### L. acidophilus NCFM grows on nutritionally relevant plant glycosides (PGs)

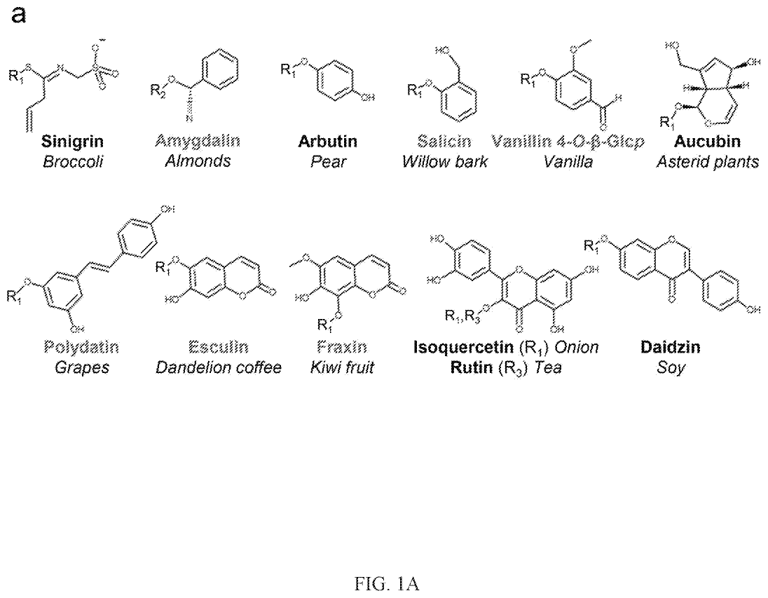

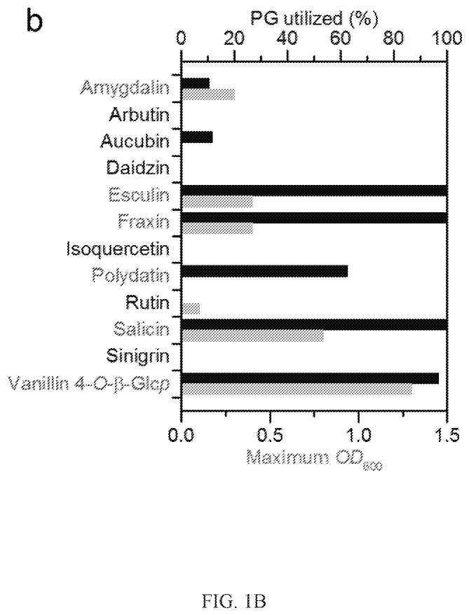

The growth of L. acidophilus NCFM was evaluated on twelve chemically diverse, nutritionally relevant and/or therapeutically active PGs 1; Table 1). FIG. 1A provides the structures and common sources of plant glycoside substrates described herein. The compounds that support growth of Lactobacillus acidophilus are in green. RI: β-D-Glcp; R2: Gentiobioside (β-D-Glcp-(1,6)-D-Glcp); R3: Rutinoside (α-L-Rhaf-(1,6)-D-Glcp). The graph in FIG. 1B depicts plant glycoside utilization analyzed by mass spectrometry and the growth as the maximum OD₆₀₀. Due to the low solubility of polydatin, OD₆₀₀ cannot be used as a growth metric and utilization of this compound is confirmed by the production of lactate as well as a high utilization level based on the metabolite analysis.

The cyanogenic di-glucoside amygdalin, coumarin glucosides esculin and fraxin, alcoholic glucoside salicin, and aldehyde glucoside vanillin 4-O-β-glucoside, all supported growth to a maximum OD₆₀₀ of 0.3-1.3 in 200 μL cultures in 96-well plates. The poor solubility of the stilbenoid polydatin precluded using OD_{600 nm} as a growth metric, but growth on this bioactive compound was verified by production of lactate and metabolite analysis. Additional Lactobacillus strains from different ecological niches were also tested for growth on the PGs amygdalin, arbutin, esculin, and salicin, as well as the control disaccharide cellobiose and glucose. L. acidophilus displayed versatile growth on PGs, together with Lactobacillus plantarum subsp. plantarum and a Lactobacillus rhamnosus strain (Table 3). Generally, the ability to grow on PGs was more common in strains isolated from the human gut niche compared to counterparts from other ecological environment,

### EXAMPLE 3

### Growth on Plant Glycosides Elicits a Prebiotic-Like Transcriptional Response in L. acidophilus NCFM

Global transcription was analyzed by RNA-sect for early-mid exponential phase of L. acidophilus NCFM growing on lactose, glucose as well as the growth-supporting PGs amygdalin, esculin, and salicin, which were selected based on availability and chemical diversity. The growth on lactose and the PGs differentially up-regulated less than 10% of the 1,832 predicted protein-coding genes as compared to glucose (Table 5). Only 2% of the genes were highly up-regulated on the PGs (Table 5). In general, differential values above 2.0 were considered relevant and indicated significant upregulation; differential values below −2.0 were considered relevant and indicated significant downregulation.

TABLE 5
Differentially upregulated genes for L. acidophilus NCFM growing on amygdalin (AM), esculin (ES), salicin (SA), lactose (Lac), glucose (Glu) and no carbon source as analyzed by RNA-Seq.
Annotation is colored according to cluster of orthologous genes classification. The differential normalized transcripts per million (TPM) ratios (DE) are colored according to level: bold (DE ≥ 4),
bold underline (DE ≥ 2), regular (1.9 ≥ DE ≥ −1.9), italicized underlined (−2 ≥ DE), italicized (−4 ≥ DE).

| | | | | | | | | | | | | | Log₂ differential | | |
|---|---|---|---|---|---|---|---|---|---|---|---|---|---|---|---|
| | | | | | | | | | Log₂ differential | | | | normalized TPM ratio | | |
| | | | Transcripts per million (rpm) | | | | | | normalized TPM ratio compared to glucose | | | | compared to lactose | | |
| Locus tag | Annotation | COG | AM | ES | SA | Lac | Glu | No carb | AM | ES | SA | Lac | AM | ES | SA |
| LBA0466 | Hypothetical | C | 2769.5 | 408.4 | 238.1 | 270 | 93.6 | 131.7 | 5.2 | 2 | 1.3 | 1.5 | 3.7 | 0.5 | -0.2 |
| | protein | | | | | | | | | | | | | | |
| LBA0887 | Hypothetical | C | 247 | 151.3 | 160.2 | 133.7 | 56.3 | 32.2 | 2.4 | 1.3 | 1.5 | 1.2 | 1.2 | 0.1 | 0.2 |
| | protein | | | | | | | | | | | | | | |
| LBA0910 | L-lactate | C | 1594.3 | 845.1 | 301.8 | 257.2 | 124.2 | 112.1 | 3.9 | 2.7 | 1.2 | 1 | 2.9 | 1.7 | 0.2 |
| | dehydrogenase | | | | | | | | | | | | | | |
| LBA1220 | Pyridine mercuric | C | 377.5 | 851.3 | 654.9 | 394.7 | 195.3 | 91.6 | 1.2 | 2 | 1.7 | 1 | 0.2 | 1.1 | 0.7 |
| | reductase | | | | | | | | | | | | | | |
| LBA1632 | NAD-dependent | C | 1223.3 | 730.5 | 300.2 | 253.3 | 70.6 | 36.7 | 4.4 | 3.3 | 2 | 1.8 | 2.6 | 1.5 | 0.2 |
| | aldehyde | | | | | | | | | | | | | | |
| | dehydrogenase | | | | | | | | | | | | | | |
| LBA1873 | Acetate kinase | C | 67.3 | 6.4 | 8.3 | 9 | 3.4 | 11.7 | 4.6 | 0.8 | 1.2 | 1.4 | 3.2 | −0.5 | −0.2 |
| LBA1878 | Glycerol kinase | C | 17.8 | 38 | 9.3 | 8.9 | 8.3 | 4.9 | 1.4 | 2.1 | 0.1 | 0 | 1.3 | 2 | 0 |
| LBA1411 | Fumarate | CS | 28.7 | 7.3 | 5.9 | 4.7 | 2.1 | 6.6 | 4 | 1.7 | 1.4 | 1.1 | 2.9 | 0.6 | 0.3 |
| | reductase | | | | | | | | | | | | | | |
| | flavoprotein | | | | | | | | | | | | | | |
| | subunit | | | | | | | | | | | | | | |
| LBA0329 | Putative cell | D | 12.3 | 88.3 | 22.1 | 22.1 | 16.3 | 6.8 | −0.1 | 2.4 | 0.4 | 0.4 | −0.5 | 1.9 | 0 |
| | division protein | | | | | | | | | | | | | | |
| LBA0197 | Oligopeptide | E | 30.6 | 126.9 | 134.5 | 13.9 | 65.6 | 2.5 | −0.8 | 0.9 | 1 | −2.3 | 1.5 | 3.1 | 3.2 |
| | binding protein | | | | | | | | | | | | | | |
| LBA0198 | Peptide binding | E | 20.2 | 50.6 | 43.2 | 5.4 | 41 | 3.9 | −0.8 | 0.2 | 0 | −3 | 2.2 | 3.2 | 3 |
| | protein | | | | | | | | | | | | | | |
| LBA0911 | Aminopeptidase | E | 1553.6 | 1141.5 | 3872 | 369.7 | 167.5 | 239.8 | 3.5 | 2.7 | 1.2 | 1.1 | 2.4 | 1.6 | 0 |
| LBA1022 | Arginase | E | 63.7 | 1342.6 | 175.5 | 291.6 | 251.2 | 323 | −1.7 | 2.3 | −0.6 | 0.2 | −1.9 | 2.1 | −0.8 |
| LBA1400 | Oligopeptide ABC | E | 43.9 | 5.2 | 94 | 22.4 | 10.8 | 0.2 | 2.3 | −1.1 | 3.1 | 1 | 1.3 | −2.1 | 2 |
| | transporter | | | | | | | | | | | | | | |
| | substrate binding | | | | | | | | | | | | | | |
| | protein | | | | | | | | | | | | | | |
| LBA1974 | Pyruvate oxidase | EH | 3124.6 | 1092.7 | 306.8 | 446.4 | 83.6 | 495.2 | 5.5 | 3.6 | 1.8 | 2.4 | 3.1 | 1.2 | −0.6 |
| LBA0160 | Anaerobic | F | 696.6 | 146 | 560.3 | 333.4 | 171.6 | 15.6 | 2.3 | −0.3 | 1.7 | 0.9 | 1.4 | −1.2 | 0.7 |
| | ribonucleoside | | | | | | | | | | | | | | |
| | triphosphate | | | | | | | | | | | | | | |
| | reductase | | | | | | | | | | | | | | |
| LBA1055 | Mutator protein | F | 45.4 | 5.8 | 14.9 | 12.1 | 14.9 | 19.9 | 1.9 | −1.4 | −0.1 | −0.3 | 2.2 | −1.1 | 0.3 |
| LBA0146 | PTS system HA | G | 74.1 | 20.2 | 25.6 | 23.8 | 7.9 | 54.6 | 3.5 | 1.3 | 1.6 | 1.6 | 1.9 | −0.3 | 0.1 |
| LBA0225 | 6-p-beta-glucosidase | G | 2089.8 | 24.4 | 25.6 | 21.5 | 27 | 28.7 | 6.5 | −0.2 | −0.1 | −0.4 | 6.9 | 0.1 | 0.2 |
| LBA0227 | Celiohiose-specific | G | 2893.2 | 6.5 | 6.9 | 4.5 | 3.5 | 3.4 | 9.9 | 0.8 | 0.9 | 0.3 | 9.6 | 0.5 | 0.6 |
| | PTS IIC | | | | | | | | | | | | | | |
| LBA0228 | Transcriptional | G | 940.7 | 27.3 | 15.6 | 15.1 | 14.1 | 16.6 | 6.3 | 0.9 | 0.1 | 0.1 | 6.3 | 0.8 | 0 |
| | regulator | | | | | | | | | | | | | | |
| LBA0455 | Mannose-specific | G | 714.4 | 1425.6 | 1007.8 | 599.9 | 282.3 | 229.1 | 1.6 | 2.3 | 1.8 | 1 | 0.6 | 1.2 | 0.7 |
| | PTS system | | | | | | | | | | | | | | |
| | component IIC | | | | | | | | | | | | | | |
| LBA0456 | Mannose-specific | G | 1293.7 | 2482.1 | 1723.5 | 1025.4 | 488.3 | 403.1 | 1.7 | 2.3 | 1.8 | 1 | 0.6 | 1.2 | 0.7 |
| | PTS system | | | | | | | | | | | | | | |
| | component IID | | | | | | | | | | | | | | |
| LBA0491 | Cellobiose-specific | G | 154.2 | 81.2 | 20.9 | 31 | 7.2 | 75.6 | 4.7 | 3.4 | 1.5 | 2.1 | 2.6 | 1.3 | −0.6 |
| | PTS IIC | | | | | | | | | | | | | | |
| LBA0503 | ABC transporter | G | 8.9 | 17.4 | 13.7 | 7.3 | 3.1 | 85.4 | 1.8 | 2.4 | 2.1 | 1.2 | 0.6 | 1.2 | 0.9 |
| | permease | | | | | | | | | | | | | | |
| LBA0505 | Fructosidase | G | 11.1 | 17.1 | 13.7 | 6.9 | 3.6 | 125 | 1.9 | 2.2 | 1.9 | 0.9 | 1 | 1.3 | 1 |
| LBA0606 | PTS system | G | 105.7 | 30.7 | 25.6 | 20.6 | 4.2 | 582.7 | 4.9 | 2.8 | 2.5 | 2.2 | 2.7 | 0.5 | 0.3 |
| | arbutin-like IIBC | | | | | | | | | | | | | | |
| | component | | | | | | | | | | | | | | |
| LBA0609 | PTS enzyme II | G | 422.6 | 370.1 | 199.6 | 153.8 | 48.5 | 4585.5 | 3.4 | 2.8 | 2 | 1.6 | 1.8 | 1.2 | 0.3 |
| | ABC component | | | | | | | | | | | | | | |
| LBA0680 | Glycogen | G | 237.7 | 168.7 | 108.3 | 131.9 | 65.5 | 597.6 | 2.1 | 1.3 | 0.7 | 1 | .2 | 0.3 | −0.3 |
| | branching enzyme | | | | | | | | | | | | | | |
| LBA0681 | Glucose-1-phosphate | G | 234.7 | 226.7 | 144.1 | 147.4 | 40.2 | 645.5 | 2.8 | 2.4 | 1.8 | 1.8 | 1 | 0.6 | −0.1 |
| | adenylyltransferase | | | | | | | | | | | | | | |
| LBA0682 | Glucose-1-phosphate | G | 319.1 | 280.1 | 201.1 | 175.4 | 49.2 | 742.3 | 3 | 2.4 | 2 | 1.8 | 1.2 | 0.6 | 0.2 |
| | adenylyltransferase | | | | | | | | | | | | | | |
| LBA0683 | Glycogen synthase | G | 426.2 | 356.1 | 283.5 | 224.9 | 58.5 | 742.7 | 3.1 | 2.5 | 2.2 | 1.9 | 1.2 | 0.6 | 0.3 |
| LBA0685 | Glycogen | G | 470.1 | 412 | 330.5 | 225.7 | 61.6 | 797.5 | 3.2 | 2.7 | 2.4 | 1.8 | 1.4 | 0.8 | 0.5 |
| | phosphorylase | | | | | | | | | | | | | | |
| LBA0686 | Amylopullulanase | G | 453.3 | 472.5 | 357.5 | 228.8 | 64.2 | 1009.4 | 3.1 | 2.8 | 2.4 | 1.8 | 1.3 | 1 | 0.6 |
| LBA0687 | Phosphoglucomutase | G | 487.4 | 532.6 | 410.2 | 409.4 | 119.8 | 1416 | 2.3 | 2.1 | 1.7 | 1.7 | 0.6 | 0.3 | 0 |
| LBA0724 | Transcription | G | 2061.6 | 1362.9 | 1214.5 | 105.4 | 29 | 44.6 | 6.4 | 5.5 | 5.3 | 1.8 | 4.6 | 3.6 | 3.5 |
| | antiterminator | | | | | | | | | | | | | | |
| LBA0725 | PTS EII system | G | 1214.2 | 1673.1 | 902.9 | 12.1 | 1.8 | 4.5 | 9.7 | 9.8 | 8.9 | 2.7 | 7 | 7.1 | 6.2 |
| LBA0726 | Phospho-beta- | G | 2460.5 | 2576.8 | 1529.5 | 133.6 | 20.1 | 28.9 | 7.2 | 6.9 | 6.2 | 2.7 | 4.5 | 4.2 | 3.5 |
| | galactosidase II | | | | | | | | | | | | | | |
| LBA0874 | Phospho-beta- | G | 116.7 | 49.2 | 28.6 | 32.5 | 14.6 | 39 | 3.3 | 1.7 | 0.9 | 1.1 | 2.2 | 0.5 | −0.2 |
| | galactosidase I | | | | | | | | | | | | | | |
| LBA0875 | Transcriptional | G | 47.5 | 31.2 | 25 | 16.3 | 11.6 | 31.5 | 2.3 | 1.3 | 1 | 0.4 | 1.9 | 0.9 | 0.6 |
| | regulator | | | | | | | | | | | | | | |
| LBA0876 | Cellobiose-specific | G | 1700.2 | 776.8 | 551. 7 | 742.4 | 97.1 | 2205 | 4.4 | 2.9 | 2.4 | 2.9 | 1.5 | 0 | −0.5 |
| | PTS IIC | | | | | | | | | | | | | | |
| LBA0877 | Cellobiose-specific | G | 1190.8 | 501.6 | 369.8 | 514.3 | 59.6 | 1998.9 | 4.6 | 3 | 2.6 | 3.1 | 1.5 | −0.1 | −0.5 |
| | PTS IIA | | | | | | | | | | | | | | |
| LBA0986 | Galactose | G | 704 | 379.4 | 240.8 | 140.8 | 92 | 137.4 | 3.2 | 2 | 1.3 | 0.6 | 2.6 | 1.4 | 0.7 |
| | mutarotase related | | | | | | | | | | | | | | |
| | ensyme | | | | | | | | | | | | | | |
| LBA1102 | Transmembrane | G | 31.2 | 226.4 | 167.6 | 90.5 | 35 | 4.7 | 0.1 | 2.6 | 2.2 | 1.3 | −1.2 | 1.3 | 0.9 |
| | protein | | | | | | | | | | | | | | |
| LBA1433 | Dihydroxyacetone | G | 318.7 | 80.6 | 39 | 35.4 | 5.8 | 100.5 | 6 | 3.7 | 2.7 | 2.6 | 3.5 | 1.1 | 0.1 |
| | kinase | | | | | | | | | | | | | | |
| LBA1434 | Dihydroxyacetone | G | 305.9 | 85.7 | 36 | 36.8 | 3.5 | 151.9 | 6.7 | 4.5 | 3.3 | 3.4 | 3.4 | 1.2 | −0.1 |
| | kinase | | | | | | | | | | | | | | |
| LBA1436 | Glycerol uptake | G | 328.8 | 84.9 | 37.6 | 37 | 2.7 | 322.4 | 7.2 | 4.9 | 3.8 | 3.8 | 3.5 | 1.1 | 0 |
| | facilitator protein | | | | | | | | | | | | | | |
| LBA1457 | Galactose-1-epimerase | G | 74.9 | 144.7 | 55.9 | 732.6 | 14.5 | 12.7 | 2.6 | 3.2 | 1.9 | 5.6 | −3 | −2.4 | −3.7 |
| LBA1458 | Galactose-1-phosphate | G | 74 | 144 | 48.4 | 679.9 | 13 | 10.1 | 2.8 | 3.4 | 1.8 | 5.7 | −2.9 | −2.3 | −3.8 |
| | uridylyltransferase | | | | | | | | | | | | | | |
| LBA1459 | Galactokinase | G | 70.3 | 123.5 | 42.2 | 610.1 | 23.9 | 7.5 | 1.8 | 2.3 | 0.8 | 4.6 | −2.8 | −2.4 | −3.9 |
| LBA1462 | Beta-galactosidase | G | 10.2 | 13 | 16.8 | 1123.2 | 16.8 | 31.6 | −0.5 | −0.5 | −0.1 | 6 | −6.5 | −6.5 | −6..1 |
| LBA1463 | Lactose permease | G | 50.7 | 47.8 | 62.2 | 1427.3 | 54.3 | 119.1 | 0.2 | −0.3 | 0.1 | 4.7 | −4.5 | −5 | −4.6 |
| LBA1467 | Beta-galactosidase | G | 42.2 | 24.1 | 14.9 | 2333 | 5 | 7.4 | 3.3 | 2.2 | 1.5 | 8.8 | −5.5 | −6.7 | −7.3 |
| | large subunit | | | | | | | | | | | | | | |
| | (lactase) | | | | | | | | | | | | | | |
| LBA1468 | Beta-galactosidase | G | 61.3 | 38.7 | 43.3 | 1790.7 | 21 | 15.9 | 1.8 | 0.8 | 1 | 6.4 | −4.6 | −5.6 | −5.4 |
| | small subunit | | | | | | | | | | | | | | |
| LBA1481 | D-ribose-binding | G | 33 | 9.2 | 6.4 | 8.1 | 4.8 | 80.7 | 3 | 0.9 | 0.4 | 0.7 | 2.3 | 0.1 | −0.4 |
| | protein precursor | | | | | | | | | | | | | | |
| LBA1482 | Ribose ABC | G | 32.6 | 8.1 | 6.1 | 7.9 | 3.8 | 71.9 | 3.4 | 1 | 0.7 | 1 | 2.3 | 0 | −0.4 |
| | transporter | | | | | | | | | | | | | | |
| LBA1483 | Ribose ABC | G | 30.8 | 4.2 | 4.2 | 6.4 | 3.1 | 47.1 | 3.6 | 0.4 | 0.4 | 1 | 2.6 | −0.7 | −0.7 |
| | transporter ATP | | | | | | | | | | | | | | |
| | binding protein | | | | | | | | | | | | | | |
| LBA1484 | D-ribose pyranase | G | 37.3 | 5.3 | 4.5 | 6.1 | 3.8 | 44.9 | 3.6 | 0.4 | 0.2 | 0.6 | 2.9 | −0.3 | −0.5 |
| LBA1485 | Ribokinase | G | 29.8 | 2.7 | 4.5 | 5.2 | 3.1 | 35.2 | 3.5 | −0.3 | 0.5 | 0.7 | 2.8 | −1 | −0.3 |
| LBA1684 | Putative PTS | G | 976.1 | 98.9 | 76.9 | 48 | 12.5 | 410.8 | 6.6 | 2.9 | 2.6 | 1.9 | 4.7 | 1 | 0.6 |
| | system IIA | | | | | | | | | | | | | | |
| | component | | | | | | | | | | | | | | |
| LBA1689 | Maltose-6′-phosphate | G | 113.8 | 12.7 | 47.4 | 9.7 | 3.5 | 352.2 | 5.3 | 1.8 | 3.7 | 1.4 | 3.9 | 0.3 | 2.2 |
| | Oucosidase | | | | | | | | | | | | | | |
| LBA1705 | PTS system IIBC | G | 18.5 | 15.9 | 8.4 | 6.1 | 3.4 | 49.5 | 2.7 | 2.2 | 1.3 | 0.8 | 1.9 | 1.3 | 0.4 |
| | component | | | | | | | | | | | | | | |
| LBA1710 | Thermostable | G | 26.7 | 9.6 | 10.8 | 9.9 | 7.4 | 17.9 | 2.1 | 0.3 | 0.5 | 0.4 | 1.7 | −0.1 | 0.1 |
| | pullulanase | | | | | | | | | | | | | | |
| LBA1812 | Alpha-glucosidase II | G | 520.3 | 120.6 | 75.4 | 68.8 | 16.2 | 32.4 | 5.3 | 2.8 | 2.2 | 2 | 3.2 | 0.8 | 0.1 |
| LBA1870 | Maltose | G | 539.7 | 109.2 | 55.6 | 46.3 | 102.1 | 19.5 | 2.7 | 0 | −0.9 | −1.2 | 3.9 | 1.2 | 0.2 |
| | phosphorylase | | | | | | | | | | | | | | |
| LBA1871 | Neopullulanase | G | 43.6 | 12.2 | 5.1 | 4.5 | 2.7 | 41.9 | 4.3 | 2.1 | 0.9 | 0.7 | 3.6 | 1.4 | 0.2 |
| LBA1872 | Oligo-1,6-glucosidase | G | 30 | 6.3 | 6.2 | 4 | 3 | 19.4 | 3.6 | 1 | 1 | 0.4 | 3.2 | 0.6 | 0.6 |
| LBA0753 | Multi-drug-type | GEPR | 26.1 | 155 | 70.4 | 29.5 | 48.1 | 12.2 | −0.6 | 1.6 | 0.5 | −0.8 | 0.1 | 2.3 | 1.2 |
| | permease | | | | | | | | | | | | | | |
| LBA1494 | Lincomycin- | GEPR | 2.6 | 69.4 | 8.2 | 5.7 | 5.3 | 6.7 | −0.7 | 3.6 | 0.6 | 0.1 | −0.8 | 3.6 | 0.5 |
| | resistance protein | | | | | | | | | | | | | | |
| LBA1653 | Nicotinamide | H | 180.2 | 996.7 | 618.8 | 369.4 | 233.8 | 37.9 | −0.1 | 2 | 1.3 | 0.6 | −0.7 | 1.4 | 0.7 |
| | mononucleotide | | | | | | | | | | | | | | |
| LBA1607 | Phosphatidylserine | I | 102.4 | 59.5 | 37.9 | 41.1 | 20.1 | 271.8 | 2.6 | 1.5 | 0.9 | 1 | 1.6 | 0.5 | −0.2 |
| | decarboxylase | | | | | | | | | | | | | | |
| | precursor | | | | | | | | | | | | | | |
| LBA0672 | Putative phosphate | J | 8689.6 | 5745.2 | 3596.1 | 2957.4 | 1599.4 | 13987.2 | 2.7 | 1.8 | 1.1 | 0.8 | 1.9 | 0.9 | 0.2 |
| | starvation | | | | | | | | | | | | | | |
| | inducible protein | | | | | | | | | | | | | | |
| | stress related | | | | | | | | | | | | | | |
| LBA1392 | Mucus binding | J | 148.6 | 537 | 114.4 | 88.9 | 99.7 | 26.8 | 0.8 | 2.3 | 0.1 | −0.2 | 1.1 | 2.5 | 0.3 |
| | protein precursor | | | | | | | | | | | | | | |
| | Mub | | | | | | | | | | | | | | |
| LBA0500 | MSM operon | K | 169.5 | 78.4 | 138.7 | 162 | 52.2 | 8.2 | 2 | 0.5 | 1.4 | 1.6 | 0.4 | −1.1 | −0.3 |
| | repressor | | | | | | | | | | | | | | |
| LBA0573 | Putative | K | 26.5 | 592 | 43.7 | 98.5 | 90.1 | 4.4 | −1.5 | 2.6 | −1.1 | 0.1 | −1.6 | 2.5 | −1.2 |
| | transcriptional | | | | | | | | | | | | | | |
| | regulator | | | | | | | | | | | | | | |
| LBA0607 | Putative | K | 252.9 | 179.3 | 86.7 | 66.9 | 47 | 1004.2 | 2.7 | 1.8 | 0.8 | 0.5 | 2.2 | 1.4 | 0.3 |
| | transcriptional | | | | | | | | | | | | | | |
| | regulator | | | | | | | | | | | | | | |
| LBA0897 | Putative Cro-like | K | 0.4 | 1.5 | 2.8 | 0.5 | 2.6 | 2.1 | −2.3 | −0.9 | 0.1 | −2.4 | 0.2 | 1.5 | 2.5 |
| | protein | | | | | | | | | | | | | | |
| LBA1021 | Putative | K | 52.4 | 797.3 | 108.6 | 186.9 | 142.7 | 181.1 | −1.2 | 2.4 | −0.5 | 0.3 | −1.5 | 2 | −0.8 |
| | transcriptional | | | | | | | | | | | | | | |
| | regulator | | | | | | | | | | | | | | |
| LBA1465 | Transcription | K | 43.7 | 47.3 | 52.9 | 485.8 | 54.6 | 1.8 | −0.1 | −0.3 | −0.1 | 3.1 | −3.2 | −3.4 | −3.2 |
| | repressor of beta- | | | | | | | | | | | | | | |
| | galactosidasegene | | | | | | | | | | | | | | |
| LBA1700 | Transcriptional | K | 96.2 | 61.9 | 23 | 28.8 | 20.9 | 126 | 2.5 | 1.5 | 0.1 | 0.4 | 2.1 | 1 | −0.4 |
| | regulator-type | | | | | | | | | | | | | | |
| LBA1701 | Melibiose operon | K | 1323.5 | 1259.8 | 84.1 | 69.9 | 39.2 | 1919.2 | 5.3 | 4.9 | 1 | 0.8 | 4.6 | 4.1 | 0.2 |
| | regulatory protein | | | | | | | | | | | | | | |
| LBA1708 | Transcriptional | K | 3.3 | 1.6 | 1.3 | 1.6 | 0.5 | 4 | 2.8 | 1.4 | 1.2 | 1.4 | 1.4 | 0 | −0.2 |
| | antiterminator | | | | | | | | | | | | | | |
| LBA1955 | Transcription | K | 5835.9 | 218.2 | 428.6 | 403.9 | 518.8 | 563.6 | 3.8 | −1.3 | −0.3 | −0.4 | 4.2 | −0.9 | 0.1 |
| | regulator family | | | | | | | | | | | | | | |
| | protein | | | | | | | | | | | | | | |
| LBA0016 | Fructokinase | KG | 387.4 | 347.8 | 300.8 | 211.4 | 90 | 410.1 | 2.4 | 1.9 | 1.7 | 1.2 | 1.2 | 0.7 | 0.5 |
| LBA0886 | Sugar kinase- | KG | 25.1 | 15.5 | 8.1 | 7.7 | 6.4 | 8.1 | 2.2 | 1.2 | 0.3 | 0.2 | 2 | 0.9 | 0 |
| | putative | | | | | | | | | | | | | | |
| | transcriptional | | | | | | | | | | | | | | |
| | regulator | | | | | | | | | | | | | | |
| LBA0545 | Hypothetical | L | 244.7 | 286.5 | 259 | 306 | 73.7 | 131.6 | 2 | 1.9 | 1.8 | 2 | 0 | −0.1 | −0.3 |
| | protein | | | | | | | | | | | | | | |
| LBA0547 | Putative site- | L | 24.9 | 50.6 | 32.5 | 37.9 | 11 | 18.6 | 1.5 | 2.1 | 1.5 | 1.7 | −0.3 | 0.4 | −0.3 |
| | specific | | | | | | | | | | | | | | |
| | recombinase | | | | | | | | | | | | | | |
| LBA1464 | Transposase | L | 133.1 | 94.4 | 182.7 | 956 | 173.7 | 30.6 | −0.1 | −1 | 0 | 2.4 | −2.5 | −3.4 | −2.4 |
| LBA1723 | Transposase | L | 176.9 | 55.8 | 75.5 | 62 | 52.1 | 94.7 | 2 | 0 | 0.5 | 0.2 | 1.8 | −0.2 | 0.2 |
| LBA1469 | UDP-glucose 4-epimerase | M | 385.3 | 385 | 235.5 | /812.3 | 85.5 | 43.2 | 2.4 | 2.1 | 1.4 | 5 | −2.6 | −2.9 | −3.6 |
| LBA1023 | Oxidoreductase | MG | 18.8 | 911.4 | 132.8 | 213.6 | 169.4 | 290.8 | −1.5 | 2.3 | −0.4 | 0.3 | −1.8 | 2 | −0.7 |
| LBA0327 | Replication protein | O | 3.5 | 43.4 | 8.1 | 9.5 | 8 | 6 | −0.9 | 2.4 | 0 | 0.2 | −1.1 | 2.1 | −0.3 |
| LBA1793 | Hypothetical | OC | 67 | 28.5 | 37.8 | 117.6 | 16.2 | 2.2 | 2.3 | 0.7 | 1.2 | 2.8 | −0.5 | −2.1 | −1.7 |
| | protein | | | | | | | | | | | | | | |
| LBA0541 | Cadmium- | P | 119.7 | 138.8 | 143.6 | 167.4 | 35.4 | 28.2 | 2 | 1.9 | 2 | 2 | −0.2 | −0.3 | −0.3 |
| | transporting | | | | | | | | | | | | | | |
| | ATPase | | | | | | | | | | | | | | |
| LBA0542 | Putative heavy- | P | 320.9 | 337.4 | 311.6 | 409.6 | 85.2 | 60.1 | 2.2 | 1.9 | 1.8 | 2.2 | 0 | −0.3 | −0.4 |
| | metal-transporting | | | | | | | | | | | | | | |
| | alpase | | | | | | | | | | | | | | |
| LBA1683 | Putative magnesium- | P | 53.6 | 20.5 | 20.2 | 14 | 15.9 | 151.4 | 2 | 0.3 | 0.3 | −0.2 | 2.3 | 0.5 | 0.5 |
| | transporting atpase | | | | | | | | | | | | | | |
| LBA0263 | Hypothetical | R | 55.9 | 59.9 | 30.2 | 27 | 16 | 40.9 | 2.1 | 1.8 | 0.9 | 0.7 | 1.4 | 1.1 | 0.1 |
| | protein | | | | | | | | | | | | | | |
| LBA0564 | HAD family | R | 4.1 | 2.8 | 1.4 | 1.5 | 1 | 3.6 | 2.3 | 1.4 | 0.4 | 0.5 | 1.8 | 0.9 | −0.2 |
| | hydrolase | | | | | | | | | | | | | | |
| LBA0604 | Immunity protein | R | 49.6 | 12.5 | 14.3 | 20.8 | 14.5 | 34 | 2 | −0.3 | −0.1 | 0.5 | 1.6 | −0.8 | −0.6 |
| LBA0605 | Putative hydrolase | R | 26.3 | 15.5 | 6.3 | 5.5 | 2.7 | 103.6 | 3.6 | 2.4 | 1.2 | 1 | 2.6 | 1.4 | 0.2 |
| LBA0728 | Hypothetical | R | 129.5 | 70.6 | 43.8 | 8.5 | 2.4 | 3.1 | 6 | 4.8 | 4.1 | 1.8 | 4.2 | 3 | 2.3 |
| | protein LBA0728 | | | | | | | | | | | | | | |
| LBA1025 | Aldo/keto | R | 7.2 | 210.8 | 23.7 | 31.7 | 30.9 | 28 | −1.8 | 2.7 | −0.4 | 0 | −1.8 | 2.7 | −0.5 |
| | reductase family | | | | | | | | | | | | | | |
| | oxidoreductase | | | | | | | | | | | | | | |
| LBA1026 | Aldo/keto | R | 8.1 | 162.5 | 26.1 | 43.3 | 31.1 | 32.9 | −1.7 | 2.3 | −0.3 | 0.4 | −2.1 | 1.9 | −0.8 |
| | reduetase family | | | | | | | | | | | | | | |
| | oxidoreductase | | | | | | | | | | | | | | |
| LBA1027 | Oxidoreductase | R | 3613.2 | 2680.9 | 2726.8 | 1733.7 | 576.2 | 85. | 2.9 | 2.1 | 2.2 | 1.5 | 1.4 | 0.6 | 0.6 |
| LBA1401 | Peroxidase (Npx) | R | 1161.2 | 609.8 | 466.6 | 294.6 | 67.4 | 184.5 | 4.4 | 3.1 | 2.7 | 2.1 | 2.3 | 1 | 0.6 |
| LBA1675 | Hypothetical | R | 1397.9 | 1975.8 | 1669.1 | 1127.6 | 400.1 | 111.9 | 2.1 | 2.2 | 2 | 1.4 | 0.6 | 0.8 | 0.5 |
| | protein | | | | | | | | | | | | | | |
| LBA1676 | ATP-dependent | R | 953.4 | 1361.3 | 1070.6 | 734.7 | 315.5 | 36.1 | 1.9 | 2 | 1.7 | 1.2 | 0.7 | 0.8 | 0.5 |
| | helicase | | | | | | | | | | | | | | |
| LBA1704 | N-Acetylmuramic | R | 23 | 21.2 | 11.6 | 10.7 | 6.6 | 67.4 | 2.1 | 1.6 | 0.7 | 0.6 | 1.4 | 0.9 | 0.1 |
| | acid-6-phosphate | | | | | | | | | | | | | | |
| | etherase | | | | | | | | | | | | | | |
| LBA1769 | Hypothetical | R | 10240.4 | 6023.9 | 5527.5 | 5636.3 | 1502.3 | 671.8 | 3 | 1.9 | 1.8 | 1.9 | 1.2 | 0 | −0.1 |
| | protein | | | | | | | | | | | | | | |
| LBA1869 | Beta-phosphoglueomutase | R | 670.5 | 156.4 | 43.1 | 43.9 | 7.8 | 67.5 | 6.7 | 4.2 | 2.4 | 2.4 | 4.2 | 1.8 | −0.1 |
| LBA0457 | Hypothetical | S | 1217.8 | 2243.1 | 1746 | 977.4 | 481.1 | 478.7 | 1.6 | 2.1 | 1.8 | 1 | 0.6 | 1.1 | 0.8 |
| | protein | | | | | | | | | | | | | | |
| LBA0555 | Myosin-crossreactive | S | 2251.5 | 186.9 | 118.8 | 165.8 | 42.9 | 655.1 | 6 | 2 | 1.4 | 1.9 | 4.1 | 0.1 | −0.5 |
| | antigen | | | | | | | | | | | | | | |
| LBA0649 | Myosin-crossreactive | S | 228.6 | 154.8 | 116.3 | 65.3 | 38 | 12.8 | 2.9 | 1.9 | 1.6 | 0.7 | 2.1 | 1.2 | 0.8 |
| | antigen | | | | | | | | | | | | | | |
| LBA0861 | Polyferredoxin | S | 153 | 785.8 | 196.3 | 272.1 | 180.1 | 179.6 | 0 | 2 | 0.1 | 0.5 | −0.5 | 1.5 | −0.5 |
| LBA1206 | Hypothetical | S | 984.2 | 636.7 | 391.2 | 302.8 | 130.9 | 1482.1 | 3.2 | 2.2 | 1.5 | 1.2 | 2 | 1 | 0.3 |
| | protein | | | | | | | | | | | | | | |
| LBA1435 | Hypothetical | S | 468.3 | 142.3 | 52.5 | 57.4 | 4.2 | 247.8 | 7.1 | 5 | 3.6 | 3.7 | 3.3 | 1.3 | −0.2 |
| | protein | | | | | | | | | | | | | | |
| LBA1656 | Hypothetical | S | 103.5 | 48.5 | 38.2 | 30.6 | 20.1 | 144 | 2.6 | 1.2 | 0.9 | 0.6 | 2.1 | 0.6 | 0.3 |
| | protein | | | | | | | | | | | | | | |
| LBA1740 | Hypothetical | S | 15.4 | 95.9 | 23.1 | 31.5 | 17.7 | 10.4 | 0.1 | 2.4 | 0.3 | 0.8 | −0.7 | 1.6 | −0.5 |
| | protein | | | | | | | | | | | | | | |
| LBA1752 | Hypothetical | S | 12.1 | 42.9 | 13 | 15.3 | 8.2 | 4.3 | 0.8 | 2.3 | 0.6 | 0.8 | 0 | 1.4 | −0.3 |
| | protein | | | | | | | | | | | | | | |
| LBA1895 | Hypothetical | S | 152.2 | 74.5 | 46.2 | 31.9 | 27.2 | 141.2 | 2.8 | 1.4 | 0.7 | 0.2 | 2.6 | 1.2 | 0.5 |
| | protein | | | | | | | | | | | | | | |
| LBA0149 | Putative | T | 1625.4 | 1325.7 | 1141 | 850.2 | 478.5 | 1432.8 | 2 | 1.4 | 1.2 | 0.8 | 1.2 | 0.6 | 0.4 |
| | nucleotide-binding | | | | | | | | | | | | | | |
| | protein | | | | | | | | | | | | | | |
| LBA0282 | Hypothetical | T | 20 | 92.4 | 34.3 | 32.7 | 20.9 | 6.7 | 0.2 | 2.1 | 0.7 | 0.6 | −0.4 | 1.4 | 0 |
| | protein | | | | | | | | | | | | | | |
| LBA0544 | Transcriptional | T | 321.3 | 342.2 | 342.8 | 416.6 | 103.3 | 120.3 | 1.9 | 1.6 | 1.7 | 2 | −0.1 | −0.3 | −0.3 |
| | regulator | | | | | | | | | | | | | | |
| LBA0574 | ABC multi drug | V | 31.9 | 490.6 | 54.6 | 109.5 | 85.3 | 7.3 | −1.2 | 2.4 | −0.7 | 0.3 | −1.5 | 2.1 | −1 |
| | exporter | | | | | | | | | | | | | | |
| LBA0575 | ABC multi drug | V | 32.6 | 381 | 55.6 | 93 | 77.3 | 9.6 | −1 | 2.2 | −0.5 | 0.2 | −1.2 | 2 | −0.8 |
| | exporter | | | | | | | | | | | | | | |
| LBA1455 | Putative surface | V | 73.9 | 28.7 | 18.3 | 53.4 | 16.9 | 212 | 2.4 | 0.7 | 0.1 | 1.6 | 0.8 | −0.9 | −1.6 |
| | layer protein | | | | | | | | | | | | | | |
| LBA1796 | ABC exporter | V | 39.3 | 35.2 | 18.3 | 52.4 | 8.8 | 5.2 | 2.4 | 1.9 | 1 | 2.5 | −0.1 | −0.6 | −1.6 |
| LBA0046 | Hypothetical | | 29.9 | 1439.6 | 270.7 | 506.3 | 292.6 | 1187.4 | −3 | 2.2 | −0.2 | 0.7 | −3.8 | 1.5 | −0.9 |
| | protein | | | | | | | | | | | | | | |
| LBA0328 | Hypothetical | — | 9 | 55.9 | 13.3 | 13.2 | 13.7 | 11.4 | −0.3 | 1.9 | −0.1 | −0.1 | −0.3 | 2 | 0 |
| | protein | | | | | | | | | | | | | | |
| LBA0330 | Hypothetical | — | 18.2 | 211.2 | 27.3 | 26.6 | 20.2 | 3.5 | 0.1 | 3.3 | 0.4 | 0.3 | −0.2 | 2.9 | 0 |
| | protein | | | | | | | | | | | | | | |
| LBA0485 | Hypothetical | — | 1655 | 868.4 | 770.3 | 1078 | 199.1 | 263.8 | 3.3 | 2 | 1.9 | 2.4 | 0.9 | −0.4 | −0.5 |
| | protein | | | | | | | | | | | | | | |
| LBA0486 | Hypothetical | — | 2443 | 1210.2 | 1205 | 1593.4 | 226 | 445.2 | 3.7 | 2.3 | 2.4 | 2.8 | 0.9 | −0.5 | −0.4 |
| | protein | | | | | | | | | | | | | | |
| LBA0487 | Hypothetical | — | 132.1 | 69.1 | 96.1 | 82.6 | 33.1 | 431.5 | 2.3 | 1 | 1.5 | 1.3 | 1 | −0.3 | 0.2 |
| | protein. | | | | | | | | | | | | | | |
| LBA0492 | Hypothetical | — | 213.1 | 108.4 | 32.8 | 44.2 | 7.9 | 97.6 | 5 | 3.7 | 2 | 2.4 | 2.6 | 1.2 | −0.5 |
| | protein | | | | | | | | | | | | | | |
| LBA0543 | Hypothetical | — | 283.1 | 278.7 | 266.3 | 345.9 | 66.2 | 65.6 | 2.4 | 2 | 2 | 2.3 | 0 | −0.4 | −0.4 |
| | protein | | | | | | | | | | | | | | |
| LBA0608 | Hypothetical | — | 239.3 | 183.9 | 95.4 | 75.1 | 34.3 | 1605.2 | 3.1 | 2.3 | 1.4 | 1.1 | 2 | 1.2 | 0.3 |
| | protein | | | | | | | | | | | | | | |
| LBA0631 | Hypothetical | — | 141786.9 | 9527.2 | 9342.6 | 10677.4 | 1183.8 | 512633.2 | 7.2 | 2.9 | 2.9 | 3.1 | 4 | −0.2 | −0.2 |
| | protein | | | | | | | | | | | | | | |
| LBA0802 | Hypothetical | — | 133.2 | 21 | 35.1 | 40.6 | 32.6 | 40.2 | 2.3 | −0.7 | 0 | 0.3 | 2 | −1 | −0.2 |
| | protein | | | | | | | | | | | | | | |
| LBA0863 | Hypothetical | — | 53.7 | 240.9 | 58.6 | 86.1 | 49.1 | 84.8 | 0.4 | 2.2 | 0.2 | 0.8 | −0.4 | 1.4 | −0.6 |
| | protein | | | | | | | | | | | | | | |
| LBA0878 | Hypothetical | — | 2.5 | 10.1 | 10.9 | 8.4 | 3.7 | 14.4 | 2 | 1.4 | 1.5 | 1.1 | 0.9 | 0.2 | 0.3 |
| | protein | | | | | | | | | | | | | | |
| LBA0888 | Hypothetical | — | 138.9 | 84.8 | 76.2 | 64 | 30 | 11.1 | 2.5 | 1.4 | 1.3 | 1 | 1.4 | 0.4 | 0.2 |
| | protein | | | | | | | | | | | | | | |
| LBA0987 | Hypothetical | — | 3.7 | 323.4 | 294.5 | 359.5 | 45.1 | 143.9 | 2.8 | 2.8 | 2.7 | 2.9 | −0.2 | 2 | −0.3 |
| | protein | | | | | | | | | | | | | | |
| LBA1019 | Mucus binding | — | 91.3 | 40.2 | 26.5 | 26.1 | 12.7 | 208.6 | 3.1 | 1.6 | 1 | 1 | 2.1 | 0.6 | 0 |
| | protein | | | | | | | | | | | | | | |
| LBA1020 | Mucus binding | — | 49.4 | 864.5 | 106.2 | 153.9 | 140.7 | 156 | −1.2 | 2.5 | −0.5 | 0.1 | −1.3 | 2.4 | −0.6 |
| | protein | | | | | | | | | | | | | | |
| LBA1227 | Hypothetical | — | 523.3 | 281.3 | 117.3 | 99.5 | 69.3 | 433.7 | 3.2 | 1.9 | 0.7 | 0.5 | 2.7 | 1.4 | 0.2 |
| | protein | | | | | | | | | | | | | | |
| LBA1370 | Putative | — | 123 | 91.2 | 73.1 | 54.3 | 37 | 13.1 | 2 | 1.2 | 0.9 | 0.5 | 1.5 | 0.7 | 0.4 |
| | bacteriophage- | | | | | | | | | | | | | | |
| | related protein | | | | | | | | | | | | | | |
| LBA1409 | Hypothetical | — | 18.3 | 16 | 7.3 | 7 | 4.4 | 17.3 | 2.3 | 1.8 | 0.7 | 0.6 | 1.7 | 1.1 | 0 |
| | protein LBA1409 | | | | | | | | | | | | | | |
| LBA1460 | Mucus binding | — | 4.5 | 5.6 | 7.9 | 186.9 | 9.8 | 5.3 | −0.9 | −0.9 | −0.4 | 4.2 | −5.1 | −5.1 | −4.6 |
| | protein precursor | | | | | | | | | | | | | | |
| LBA1461 | Putative regulator | — | 2.3 | 3 | 7.3 | 168.9 | 5.5 | 4.4 | −1 | −1 | 0.3 | 4.9 | −5.9 | −5.9 | −4.6 |
| LBA1539 | Hypothetical | — | 47.1 | 59.4 | 86.5 | 160.7 | 23.2 | 15.8 | 1.3 | 1.3 | 1.8 | 2.7 | −1.5 | −1.5 | −0.9 |
| | protein | | | | | | | | | | | | | | |
| LBA1608 | Hypothetical | — | 139.8 | 63.4 | 46.3 | 34.4 | 20.8 | 295.2 | 3 | 1.5 | 1.1 | 0.7 | 2.3 | 0.8 | 0.4 |
| | protein | | | | | | | | | | | | | | |
| LBA1611 | Surface protein | — | 290.9 | 1213 | 294.6 | 119.5 | 159.4 | 15.8 | 1.1 | 2.8 | 0.8 | −0.5 | 1.6 | 3.3 | 1.3 |
| LBA1612 | Fibrinogen-binding protein | — | 270.7 | 965.4 | 238.7 | 144.9 | 132.6 | 11.4 | 1.3 | 2.8 | 0.8 | 0.1 | 1.2 | 2.7 | 0.7 |
| LBA1633 | Surface protein | — | 29.1 | 7.8 | 14.1 | 17.3 | 7.3 | 16.5 | 2.3 | 0 | 0.9 | 1.2 | 1.1 | −1.2 | −0.3 |
| LBA1652 | Mucus binding | — | 73.8 | 34.6 | 23.6 | 17.8 | 11.4 | 7.9 | 3 | 1.5 | 1 | 0.6 | 2.4 | 0.9 | 0.4 |
| | protein precursor | | | | | | | | | | | | | | |
| | Mub | | | | | | | | | | | | | | |
| LBA1655 | Hypothetical | — | 14.5 | 5.3 | 6.1 | 5.1 | 4.1 | 3.4 | 2.1 | 0.3 | 0.5 | 0.3 | 1.8 | 0 | 0.2 |
| | protein | | | | | | | | | | | | | | |
| LBA1686 | Hypothetical | — | 826.6 | 167 | 100.9 | 148.3 | 176.6 | 300.5 | 2.5 | −0.2 | −0.9 | −0.3 | 2.8 | 0.1 | −0.6 |
| | protein | | | | | | | | | | | | | | |
| LBA1693 | Hypothetical | — | 57.4 | 523 | 120.8 | 220.4 | 107 | 65.9 | −0.6 | 2.2 | 0.1 | 1 | −1.6 | 1.2 | −0.9 |
| | protein | | | | | | | | | | | | | | |
| LBA1694 | Hypothetical | — | 28.8 | 697.9 | 74.1 | 171.1 | 82.5 | 172 | −1.3 | 3 | −0.2 | 1 | −2.3 | 2 | −1.2 |
| | protein | | | | | | | | | | | | | | |
| LBA1709 | Mucus binding | — | 48.2 | 33.7 | 3.9 | 3.7 | 2.5 | 9 | 4.5 | 3.6 | 0.5 | 0.5 | 4 | 3.2 | 0.1 |
| | protein precursor | | | | | | | | | | | | | | |
| | Mub | | | | | | | | | | | | | | |
| LBA1784 | Hypothetical | — | 107.9 | 143.4 | 58.6 | 170.8 | 41.9 | 26.3 | 1.6 | 1.7 | 0.4 | 2 | −0.3 | −0.3 | −1.6 |
| | protein | | | | | | | | | | | | | | |
| LBA1786 | Hypothetical | — | 69.4 | 86.6 | 38.1 | 111.3 | 23.7 | 10.8 | 1.8 | 1.8 | 0.6 | 2.2 | −0.4 | −0.4 | −1.6 |
| | protein | | | | | | | | | | | | | | |
| LBA1791 | Hypothetical | — | 44.6 | 39.7 | 20.1 | 84.6 | 17.7 | 2.2 | 1.6 | 1.1 | 0.1 | 2.2 | −0.6 | −1.1 | −2.1 |
| | protein | | | | | | | | | | | | | | |
| LBA1792 | Hypothetical | — | 36.6 | 26.1 | 21.9 | 73.8 | 14.1 | 2.3 | 1.6 | 0.8 | 0.6 | 2.3 | −0.7 | −1.6 | −1.8 |
| | protein | | | | | | | | | | | | | | |
| LBA1794 | Gassericin K7 B | — | 54.4 | 53.6 | 20.8 | 70.8 | 14.5 | 2.6 | 2.2 | 1.8 | 0.5 | 2.2 | −0.1 | −0.5 | −1.8 |
| | accessory protein | | | | | | | | | | | | | | |
| LBA1797 | Hypothetical | — | 2913.5 | 816 | 326.4 | 3776.4 | 131.3 | 37.7 | 4.7 | 2.6 | 1.3 | 4.8 | −0.1 | −2.3 | −3.6 |
| | protein | | | | | | | | | | | | | | |
| LBA1801 | Hypothetical | — | 30.2 | 8.5 | 7.6 | 23.8 | 4.2 | 3 | 3.1 | 0.9 | 0.8 | 2.5 | 0.7 | −1.5 | −1.7 |
| | protein | | | | | | | | | | | | | | |
| LBA1802 | Hypothetical | — | 55.9 | 15.5 | 12.4 | 44.3 | 7.4 | 3.5 | 3.2 | 1 | 0.7 | 2.5 | 0.6 | −1.6 | −1.9 |
| | protein | | | | | | | | | | | | | | |
| LBA1803 | Hypothetical | — | 81.4 | 26.6 | 21.6 | 109.5 | 12.9 | 15.7 | 2.9 | 1 | 0.7 | 3 | −0.1 | −2.1 | −2.4 |
| | protein. | | | | | | | | | | | | | | |
| LBA1804 | Hypothetical | — | 162.2 | 57.2 | 30.7 | 246.4 | 18.2 | 12.8 | 3.4 | 1.6 | 0.7 | 3.7 | −0.3 | −2.2 | −3 |
| | protein | | | | | | | | | | | | | | |
| LBA1805 | Hypothetical | — | 319.7 | 101.7 | 61.1 | 452.7 | 30.4 | 24.6 | 3.7 | 1.7 | 1 | 3.9 | −0.2 | −2.2 | −2.9 |
| | protein | | | | | | | | | | | | | | |
| | Hypothetical | — | 35.4 | 85.5 | 25.3 | 21.6 | 18.7 | 7.3 | 1.2 | 2.1 | 0.4 | 0.2 | 1 | 1.9 | 0.2 |
| | protein | | | | | | | | | | | | | | |

Of the up-regulated genes, 55 were shared by two or more of the PGs, whereas 58, 35, and 0 were uniquely induced for amygdalin, esculin, and salicin, respectively, indicating a more extensive and unique cellular responses to amygdalin and to a less extent esculin as compared to salicin. Amygdalin, which supported the lowest growth, interestingly up-regulated the highest number of genes (116 genes), followed by esculin (87) and salicin (33). Carbohydrate metabolism and transport genes comprised about one third of the differential transcriptome. The transcriptional response also revealed the up-regulation of genes encoding proteins that are predicted to be associated with mucus, fibrinogen and epithelial cell adhesion (e.g., LBA0649, LBA1392, LBA1633, and LBA1709; Table 5 and Table 6).

TABLE 6
Highly up-regulated genes in the transcriptome of L. acidophilus NCFM growing on amygdalin (Amy),
esculin (Esc), or salicin (Sal). The included genes display a log₂ differential expression ratio of
the normalized transcripts per million (nTPM) ≥4 for the plant glycosides as compared to glucose (Glc).

| | | | Log₂ Ratio | Log₂ Ratio | Log₂ Ratio |
|---|---|---|---|---|---|
| Locus tag | Annotation* | COG^{†} | Amy/Glc^{‡} | Esc/Glc^{‡} | Sal/Glc^{‡} |
| LBA0227 | PTS EIIC | G | 9.9 | 0.8 | 0.9 |
| LBA0725 | PTS EIICBA | G | 9.7 | 9.8 | 8.9 |
| LBA0726 | Phospho-β-glucosidase (GH1) | G | 7.2 | 6.9 | 6.2 |
| LBA1436 | Glycerol uptake facilitator protein | G | 7.2 | 4.9 | 3.8 |
| LBA0631 | Hypothetical protein | — | 7.2 | 2.9 | 2.9 |
| LBA1435 | Hypothetical protein | S | 7.1 | 5 | 3.6 |
| LBA1434 | Dihydroxyacetone kinase | G | 6.7 | 4.5 | 3.3 |
| LBA1869 | β-Phosphoglucomutase | R | 6.7 | 4.2 | 2.4 |
| LBA1684 | PTS EIIA | G | 6.6 | 2.9 | 2.6 |
| LBA0225 | Phospho-β-glucosidase (GH1) | G | 6.5 | −0.2 | −0.1 |
| LBA0724 | Transcriptional regulator (antiterminator) | K | 6.4 | 5.5 | 5.3 |
| LBA0228 | Transcriptional regulator | G | 6.3 | 0.9 | 0.1 |
| LBA1433 | Dihydroxyacetone kinase | G | 6 | 3.7 | 2.7 |
| LBA0728 | Hypothetical protein | R | 6 | 4.8 | 4.1 |
| LBA0555 | Myosin-cross-reactive antigen/Fatty acid | S | 6 | 2 | 1.4 |
| | hydratase | | | | |
| LBA1974 | Pyruvate oxidase | E | 5.5 | 3.6 | 1.8 |
| LBA1689 | Maltose-6′-phosphate glucosidase (GH4) | G | 5.3 | 1.8 | 3.7 |
| LBA1812 | α-Glucosidase II (GH31) | G | 5.3 | 2.8 | 2.2 |
| LBA1701 | Melibiose operon regulatory protein | K | 5.3 | 4.9 | 1 |
| LBA0466 | Phosphoenolpyruvate carboxykinase (ATP) | C | 5.2 | 2 | 1.3 |
| LBA0492 | Hypothetical protein | — | 5 | 3.7 | 2 |
| LBA0606 | PTS EIIBC | G | 4.9 | 2.8 | 2.5 |
| LBA0491 | PTS EIIC | G | 4.7 | 3.4 | 1.5 |
| LBA1797 | Hypothetical protein | — | 4.7 | 2.6 | 1.3 |
| LBA0877 | PTS EIIA | G | 4.6 | 3 | 1.3 |
| LBA1873 | Acetate kinase | C | 4.6 | 0.8 | 1.2 |
| LBA1709 | Mucus binding protein precursor | — | 4.5 | 3.6 | 0.5 |
| LBA1632 | NAD-dependent aldehyde dehydrogenase | C | 4.4 | 3.3 | 2 |
| LBA1401 | Peroxidase (Npx) | R | 4.4 | 3.1 | 2.7 |
| LBA0876 | PTS EIIC | G | 4.4 | 2.9 | 2.4 |
| LBA1871 | Neopullulanase (GH13) | G | 4.3 | 2.1 | 0.9 |
| LBA1411 | Fumarate reductase flavoprotein subunit | C | 4 | 1.7 | 1.4 |

*Annotation based on homology or functional characterization when possible.
^{†}COG: Clusters of Orthologous Genes classification; C: Energy production and conversion; E; Amino acid metabolism and transport; G: Carbohydrate metabolism and transport; K: Transcription; R: General functional prediction only; and S: Function unknown.
^{‡}Differential transcription log₂ ratio of normalized transcripts per million relative to glucose.

Interestingly, genes encoding cellular defense redox enzymes, e.g. a peroxidase (LBA1401) and an oxidoreductase (LBA1025), were also up-regulated, indicating possible xenobiotic stress response (Table 5 and. Table 6). Multi-drug efflux ABC export systems were also upregulated (e.g., LBA0574-0575, together with 41 hypothetical proteins; Table 5). Altogether, growth on PGs appeared to promote traits associated with probiotic action, through increased host interaction and adhesion, which was also observed for L. rhamnosus after pretreatment with the PGs rutin and phloridzin.

### EXAMPLE 4

### Specific PTS Uptake Systems and Specialized phospho-β-glucosidases are Essential for Growth on Plant D-Glucosides

Two gene loci were highly differentially up-regulated upon growth on PGs (Table 5 and Table 6), which was also corroborated with qRT-PCR analysis (data not shown). The first locus encompassed four genes, which were highly up-regulated (log₂ ratio 4.1-8.9, corresponding to 17-478 folds upregulation) for all three PGs. These genes encode a LicT transcriptional antiterminator (LBA0724), an EIICBA component of a PTS system (LBA0725), a phospho-β-glucosidase (P-Bgl; LBA0726) of glycoside hydrolase family 1 (GH1) according to the CAZy database, and a hypothetical protein (LBA0728). FIG. 2 provides the transcriptional profiles and conservation of plant glycoside utilization loci. The RNA read coverage for amygdalin (dark green), esculin (light green), salicin (turquoise) and glucose (light gray). FIG. 2A shows the top upregulated locus in L. acidophilus NCFM on the three plant glycosides, encodes a transcriptional regulator (LBA0724), a PTS EIIBCA transporter (LBA0725), and a phospho-β-glucosidase (P-Bgl) of glycoside hydrolase family 1 (GH1) (LBA0726), and a hypothetical protein (white). FIG. 2B provides a locus upregulated exclusively on amygdalin also encodes a P-Bgl (LBA0225), and a PTS EIIC transporter (LBA0227). Conservation of the loci in selected lactobacilli from the delbrueckii group and the amino acid sequence identities compared to L. acidophilus NCFM are shown. The red vertical line signifies scaffold border. Predicted rho-independent transcriptional terminators are shown as hairpin loops with overall confidence scores (ranging from 0 to 100),

These genes, except the less transcribed LBA0728 that belongs to the Lactobacillus core genome, are among the top 10% most upregulated genes in the PG transcriptomes (Table 5). The second locus, which was only transcriptionally responsive to amygdalin, encodes another P-Bgl of GH1 (LBA0225), a divergently transcribed PTS EIIC component (LBA0227) and a transcriptional regulator (LBA0228) (FIG. 2B). Both these gene loci are strictly conserved in the L. acidophilus species, and to some extent in related lactobacilli from the delbrueckii group (FIG. 2; Table 7).

TABLE 7
Conservation of plant glycoside utilization gene loci identified in this work (annotated by their locus tags and accession numbers)
in Lactobacillus acidophilus strains in the NCBI organism database.

| | | | | | | ID as compared to L. acidophilus NCFM genes (loci and accession no.) | | | | | | | |
|---|---|---|---|---|---|---|---|---|---|---|---|---|---|
| | | | | | | LBA0225 | LBA0226 | LBA0227 | LBA0228 | LBA0724 | LBA0725 | LBA0726 | LBA0728 |
| | | Size | Scaf- | | Pro- | AAV421 | AAV421 | AAV421 | AAV421 | AAV425 | AAV4259 | AAV425 | AAV425 |
| Strain | BioProject | (Mb) | folds | Genes | teins | 20.1 | 21.1 | 22.1 | 23.1 | 95.1 | 6.1 | 97.1 | 98.1 |
| NCFM | PRJNA82 | 1.99 | 1 | 1927 | 1832 | 100% | 100% | 100% | 100% | 100% | 100% | 100% | 100% |
| La-14 | PRJNA196170 | 1.99 | 1 | 1948 | 1835 | 100% | 100% | 100% | 100% | 100% | 99% | 100% | 100% |
| | | | | | | | | | | (Cover: | | | |
| | | | | | | | | | | 99%) | | | |
| FSI4 | PRJNA271341 | 1.99 | 1 | 1948 | 1845 | 100% | 100% | 100% | 100% | 100% | 99% | 100% | 100% |
| | | | | | | | | | | (Cover: | | | |
| | | | | | | | | | | 99%) | | | |
| ATCC | PRJNA31477 | 2.02 | 38 | 1957 | 1802 | 100% | 100% | 100% | 99% | 100% | 99% | 100% | 99% |
| 4796 | | | | | | (Cover: | | | | (Cover: | | | |
| | | | | | | 71%) | | | | 99%) | | | |
| CIP | PRJEB1532 | 1.95 | 34 | 1935 | 1779 | 100% | 100% | 100% | 100% | 100% | 99% | 100% | 100% |
| 76.13 | | | | | | | | | | (Cover: | | | |
| | | | | | | | | | | 99%) | | | |
| DSM | PRJEB18139 | 1.99 | 27 | 1944 | 1827 | 100% | 100% | 99% | 100% | 100% | 99% | 100% | 100% |
| 9126 | | | | | | | | | | (Cover: | | | |
| | | | | | | | | | | 99%) | | | |
| CIRM- | PRJEB1531 | 2.00 | 22 | 1937 | 1819 | 100% | 100% | 100% | 100% | 99% | 99% | 1000/ | 1000/ |
| BIA | | | | | | | | | | | | | |
| 445 | | | | | | | | | | | | | |
| DSM | PRJNA222257 | 1.95 | 30 | 1913 | 1787 | 100% | 100% | 100% | 100% | 100% | 99% | 100% | 100% |
| 20079 | | | | | | | | | | (Cover: | | | |
| | | | | | | | | | | 99%) | | | |
| DSM | PRJEB1533 | 2.05 | 21 | 1987 | 1865 | 100% | 100% | 100% | 100% | 99% | 99% | 100% | 100% |
| 20242 | | | | | | | | | | | | | |
| CIRM- | PRJEB1530 | 1.99 | 19 | 1947 | 1841 | 100% | 100% | 100% | 100% | 100% | 99% | 100% | 100% |
| BIA | | | | | | | | | | (Cover: | | | |
| 442 | | | | | | | | | | 99%) | | | |
| ATCC | PRJNA263693 | 1.96 | 20 | 1914 | 1780 | 100% | 100% | 100% | 100% | 100% | 99% | 100% | 100% |
| 4356 | | | | | | | | | | (Cover: | | | |
| | | | | | | | | | | 99%) | | | |
| WG- | PRJNA317797 | 1.95 | 74 | 1944 | 1815 | 100% | 100% | 100% | 100% | 100% | 99% | 100% | 100% |
| LB4V | | | | | | | | | | | | | |

Table 7 shows the amino acid identities and the sequence coverage if it is less than 100% of the protein in L. acidophilus NCFM.

To establish the functional significance of these two loci, single deletions of each PTS EII and P-Bgl gene, and a double deletion of both P-Bgl genes were constructed using the upp-based counter-selectable gene replacement system (Table 2) and the growth phenotypes of the mutant strains were analyzed (FIG. 3). Phenotypic growth analyses of deletion mutants of EII PTS transporters (FIGS. 3A, 3C, 3E, 3G, and 3I) and phospho-β-glucosidase (FIGS. 3B, 3D, 3F, 3H, and 3J) on the β-glucosides esculin, salicin, amygdalin and the disaccharides gentiobiose and cellobiose. The background Aupp strain is shown as a grey fill graph and the growth of the mutant strains is shown as: PTS EIIC (LBA0227, pink triangle), the phospho-β-glucosidase (LBA0225, light blue triangles), the PTS EIICBA (LBA0725, yellow squares), the second phospho-β-glucosidase (LBA0726, lilac squares) and the double phospho-β-glucosidase mutant (LBA0225/LBA0726, black stars). The color scheme is consistent with that used for the gene loci in FIG. 2.

The growth of the ΔLBA0725 mutant (inactive PTS EIICBA) was abolished on esculin and salicin, severely reduced on amygdalin, and moderately reduced on cellobiose and gentiobiose. The abolished growth on esculin and salicin identifies this EIICBA as the sole transporter for these PGs, but the reduced growth on the other compounds suggests additional roles of this transport system. The growth profile of the ΔLBA0726 mutant lacking a functional P-Bgl was similar for the PGs, but the growth on both cellobiose and gentiobiose was unaffected. This phenotype also supports the exquisite specificity of the P-Bgl (LBA0726) towards the PGs esculin and salicin (FIG. 3). Accordingly, the specificity of this locus can be assigned to the uptake and hydrolysis of PGs with a preference for distinct mono-glucosylated small aromatic aglycones.

The growth of the ΔLBA0227 mutant (inactive EIIC) in the second locus, which was exclusively up-regulated by amygdalin, was abolished on both amygdalin and gentiobiose (FIGS. 3E, 3G), both sharing a β-(1,6)-di-glucoside moiety (FIG. 1). The phenotypes for salicin and esculin were invariant (FIGS. 3A, 3C). These data provide compelling evidence for the specificity of this PTS EIIC transporter for amygdalin and gentiobiose, consistent with the previously reported up-regulation in response to gentiobiose. This specificity is also supported by the phenotype of the P-Bgl mutant (ΔLBA0225). The severe reduction in growth for the ΔLBA0726 mutant lacking the P-Bgl from the first locus on amygdalin (FIG. 3F), but not on gentiobiose (FIG. 3H), suggests a role of this enzyme in the catabolism of amygdalin. Indeed, growth on amygdalin is only abolished with the double P-Bgl mutant (FIG. 3F). The identification of low levels of prunasin, the singly deglucosylated form of amygdalin (Table 8) suggests that the deglycosylation of amygdalin occurs in two steps with sequential cleavage of the non-reducing β-(1,6)-linked glucosyl by the P-Bgl that recognizes the β-(1,6)-gentiobiose moiety (LBA0225) and by the second P-Bgl, which cleaves mono-glucosylated compounds (LBA0726) to release the aglycone moiety.

TABLE 8
Plant glycosides and their metabolites in L. acidophilus NCFM culture supernatants as analyzed by
UHPLC-qTOF-MS. The starting plant glycoside substrates. which were identified in the cultures before
inoculation. are in bold and underlined. The aglycones are in bold and the metabolite analyses were
carried out from the 24 hours culture supernatant samples.

| | | | UV | MS | | | | |
|---|---|---|---|---|---|---|---|---|
| | | | Rt^{†} | R^{‡} | Primary | Calc. | Identified | |
| Sample* | Compound ID | Formula | (min) | (min) | ion mode | m/z | m/z | ppm |
| Amy | Amygdalin^{§} | C₂₀H₂₇NO₁₁ | 3.45 | 3.535 | [M + HCOO]⁻ | 502.1566 | 502.1569 | 0.3 |
| Amy | Benzaldehyde^{§} | C₇H₆O | 6.04 | ND^{¶} | — | — | — | — |
| Amy | Prunasin | C₁₄H₁₇NO₆ | ND | 4.163 | [M + HCOO]⁻ | 340.1038 | 340.1032 | 2.01 |
| Arb | Arbtatin^{§} | C₁₂H₁₆O₇ | 1.98 | 2.055 | [M + HCOO]⁻ | 317.0878 | 317.088 | 0.17 |
| Auc | Aucubin | C₁₅H₂₂O₉ | ND | 2.00^{#} | [M + HCOO]⁻ | 391.1246 | 391.1252 | 1.28 |
| Esc | Esculin^{§} | C₁₅H₁₆O₉ | 2.94 | 3.008 | [M − H]⁻ | 339.0722 | 339.0726 | 1.02 |
| Esc | Esculetin^{§} | C₉H₆O₄ | 3.59 | 3.666 | [M − H]⁻ | 177.0193 | 177.0196 | 1.25 |
| Esc | Scopeletin | C₁₀H₈O₄ | 4.86 | 4.719 | [M − H]⁻ | 191.0350 | 191.0345 | 3.28 |
| Fra | Fraxin | C₁₆H₁₈O₁₀ | 3.47 | 3.525 | [M − H]⁻ | 369.0827 | 369.083 | 0.27 |
| Fra | Fraxetin | C₁₀H₈O₅ | 4.04 | 4.120 | [M − H]⁻ | 207.0299 | 207.03 | 0.06 |
| Fra | Esculin^{§} | C₁₅H₁₆O₉ | 2.96 | 3.013 | [M − H]⁻ | 339.0722 | 339.0719 | 1.19 |
| Fra | Esculetin^{§} | C₉H₆O₄ | 3.62 | 3.678 | [M − H]⁻ | 177.0193 | 177.019 | 2.79 |
| Fra | Scopeletin | C₁₀H₈O₄ | 4.86 | 4.869 | [M − H]⁻ | 191.0350 | 191.0352 | 0.77 |
| IQ | Isoquercetin | C₂₁H₂₀O₁₂ | 4.31 | 4.382 | [M − H]⁻ | 463.0882 | 463.0884 | 0.14 |
| PD | Polydatin | C₂₀H₂₂O₈ | 4.37 | 4.434 | [M + HCOO]⁻ | 435.1297 | 435.1299 | 0.46 |
| PD | Resveratrol | C₁₄H₁₂O₃ | 5.69 | 5.764 | [M − H]⁻ | 227.0714 | 227.0716 | 0.13 |
| Rut | Rutin | C₂₇H₃₀O₁₆ | 4.08 | 4.15 | [M − H]⁻ | 609.1461 | 609.1467 | 0.76 |
| Sal | Salicin^{§} | C₁₃H₁₈O₇ | 2.79 | 2.865 | [M + HCOO]⁻ | 331.1035 | 331.1032 | 1.17 |
| Sal | Salicyl alcohol^{§} | C₇H₈O₂ | 3.52 | 3.592 | [M − H]⁻ | 123.0452 | 123.0458 | 0.88 |
| Van | Vanillin 4-O-β-Glcp | C₁₄H₁₈O₈ | 3.12 | 3.178 | [M + HCOO]⁻ | 359.0984 | 359.0984 | 0.86 |
| Van | Vanillin | C₈H₈O₃ | 4.61 | 4.683 | [M − H]⁻ | 151.0401 | 151.0401 | 0.34 |

*Supernatant of L. acidophilus NUM growing on amygdalin (Amy), arbutin (Arb), aucubin (Auc), esculin (Esc), fraxin (Fra), polydatin (PD), isoquercetin (IQ), rutin (Rut), salicin (Sal), or vanillin 4-O-β-glucoside (Van).
^{†}Retention time measured in the UV detector.
^{‡}Retention time in the MS detector.
^{§}Confirmed by comparison with standard compounds.
^{¶}Not detected.
^{#}Compound eluted in several peaks, where the predominant one is noted.

Based on these data, the specificity of the locus encoding the PTS EIIC transporter (LBA0227) and the phospho-β-glucosidase (LBA0225) can be assigned to compounds with a β-(1,6)-di-glucoside motif like gentiobiose and amygdalin. The full deglycosylation of PGs possessing a gentiobiose moiety like amygdalin, however, requires the additional activity of the second P-Bgl (LBA0726).

### EXAMPLE 5

L. acidophilus prefers PGs that support highest growth and externalizes bioactive aglycones

The growth of L. acidophilus NCFM was monitored and analyzed the metabolites in the culture supernatants at 0 and 24-h. The PGs were all identified in the pre-culture medium (Table 8). Depletion of the PGs that supported growth (FIG. 1) was proportional to growth (final OD₆₀₀) and the respective aglycones lacking the glucosyl moiety (loss of 162 Da, Table 8) were identified in the culture supernatants. The growth on polydatin was verified from the extent of depletion (FIG. 1), the identification of the aglycone resveratrol (Table 8), and the production of lactate. The only deviation from this trend was the absence of the aglycone of amygdalin (mandelonitrile). Instead, the main metabolite of amygdalin utilization was benzaldehyde, which was only detectable by UV due to its volatility. The PGs that did not support growth, persisted and no metabolites were detected at 24 h.

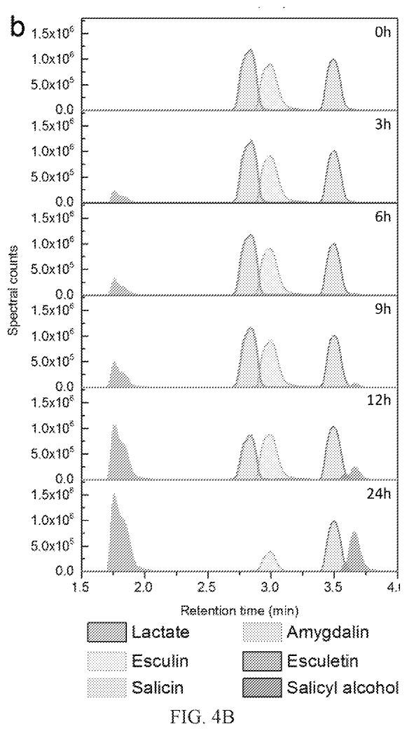

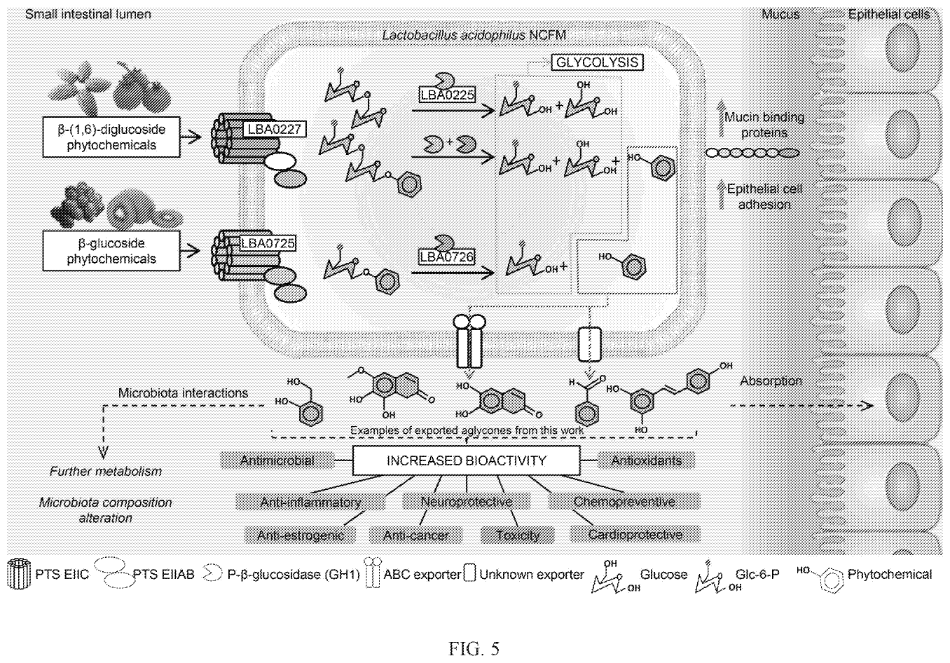

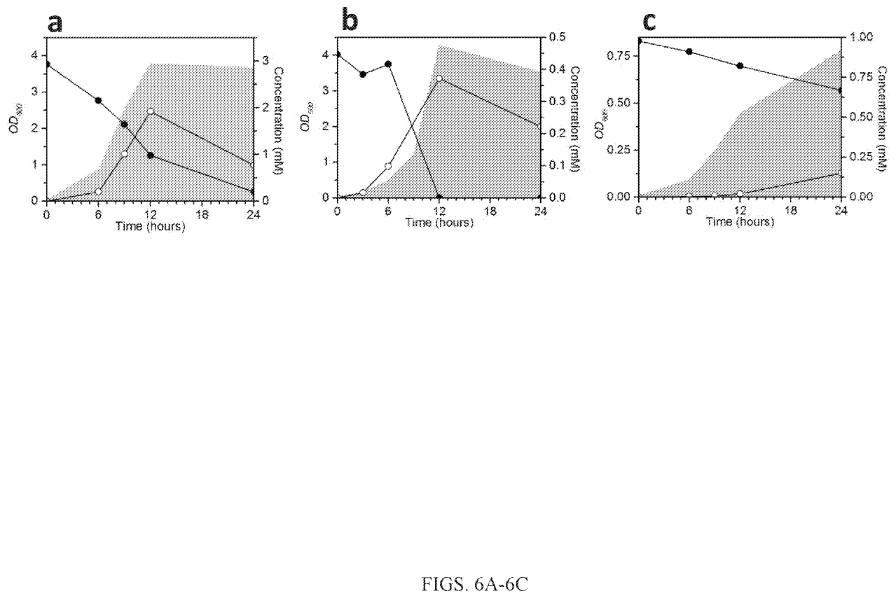

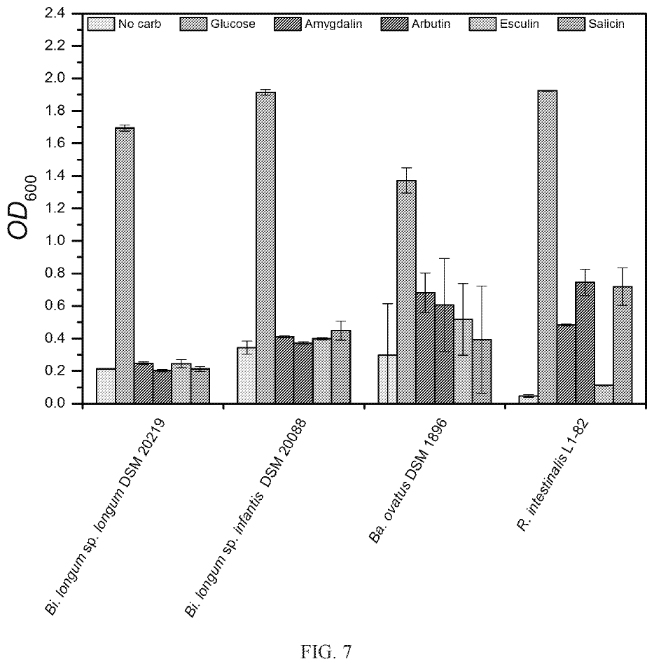

The temporal change in concentrations of the three most available PGs-salicin, esculin and amygdalin and their metabolites in culture supernatants were also monitored. The concentration of salicin decreased throughout the growth period (FIG. 4A and FIG. 6), while an inverse trend was observed for the aglycone salicyl alcohol during the exponential phase. FIG. 4 provides time-resolved metabolite analysis of L. acidophilus NCFM growing on plant glucosides. Time course depletion of salicin and appearance of its aglycone salicin alcohol in the culture supernatants is visualized as the area under the A270 nm peaks in the UHPLC-qTOF-MS chromatograms in FIG. 4A, Preference of L. acidophilus NUM to plant glycosides during growth on an equimolar mixture of salicin, esculin and amygdalin is shown in FIG. 4B. Salicin is preferred followed by esculin, while amygdalin is hardly consumed after 24 h. The aglycones of the plant glycosides and the concentration of lactate increase concomitant with growth. Notably, the aglycone moiety of salicin per se was unable to support growth of L. acidophilus (data not shown). The same pattern was observed for esculin, which was depleted concomitantly with the increase in concentration of the aglycone metabolite esculetin during the first 12 hours of growth (FIG. 6).

The concentration of amygdalin in the culture supernatant also decreased steadily concomitant with an increase in benzaldehyde (FIG. 6). In contrast to the other two PGs, however, only about a third of the initial amygdalin was utilized during 24 hours growth and the summed concentration of amygdalin and benzaldehyde was invariant over time. Low levels of the mono-deglucosylated metabolite of amygdalin, i.e. prunasin, were identified (Table 8). Although the aglycone, mandelonitrile, was identified in the first six hours ([M+CH₃COO]⁻ adduct, m/z 192.0664), the main amygdalin metabolite was benzaldehyde, which is produced by hydrogen cyanide elimination reaction of mandelonitrile. This reaction is catalyzed by nitrite lyase, but is also reported to occur spontaneously. This is the likely scenario in this analysis, as no nitrile lyase genes are encoded by L. acidophilus. Detection of traces of scopoletin, the methylated form of the esculin aglycone (Table 8), is the only evidence for enzymatic modification of the aglycones of PGs, but the paucity of this species sheds doubt on the specificity of this modification. Taken together, metabolite analyses are supportive of L. acidophilus largely exporting non-carbohydrate moieties without enzymatic modification. The mechanism of externalization is not clear, but export systems (e.g., an ATP-binding cassette exporter in the case of esculin (LBA0573-5)) are upregulated in the transcriptome (Table 5).

To evaluate whether amygdalin, esculin, and salicin are taken up randomly or according to a certain preference, the supernatants of L. acidophilus NCFM grown on equimolar concentrations of these PGs were analyzed. Strikingly, salicin was the first compound to be fully depleted, followed by esculin, whereas significant amounts of amygdalin persisted after 24 hours growth (FIG. 4B), which established a clear preference of L. acidophilus in the utilization of PGs that supports best growth.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clause:

Clause 1. A composition comprising a probiotic bacterial strain, a prebiotic plant glycoside, and a physiologically acceptable carrier and/or excipient, wherein the probiotic bacterial strain is capable of converting the prebiotic plant glycoside into a bioactive aglycone, or derivative thereof.

Clause 2. The composition of clause 1, wherein the probiotic bacterial strain comprises a bacterial species from the genus Lactobacillus,

Clause 3. The composition of clause 2, wherein the bacterial species is L. acidophilus, L. amylovorus, L. animalis, L. crispatus, L. fermentum, L. gasseri, L. helveticus, L. intestinalis, L. jensenii, L. johnsonii, L. plantarum, L. reuteri, L. rhamnosus, and combinations thereof.

Clause 4. The composition of clause 3, wherein the bacterial strain is selected from the group consisting of L. acidophilus LA-1, L. acidophilus NCFM, L. antylovorus (ATCC 33620, DSM 20531). L. animalis (DSM 20602), L. crispatus (ATCC 33820, DSM 20584). L. fermentum (ATCC 14931), L. gasseri (ATCC 33323), L. helveticus CNRZ32, L. intestinalis Th4 (ATCC 49335, DSM 6629), L. jensenii (ATCC 25258, 62G, DSM 20557), L. johnsonii (ATCC 33200). L. plantarum sp. plantarum (ATCC 14917. LA70), L. reuteri (ATCC 23272, DSM 20016), L. rhamnosus GG (ATCC 53103), and combinations thereof.

Clause 5. The composition of clause 4, wherein the bacterial strain is L. acidophilus NCFM.

Clause 6. The composition of any one of clauses 1 to 5, further comprising at least a second probiotic bacterial strain that is not a bacterial species from the genus Lactobacillus.

Clause 7. The composition of clause 6, wherein the at least second probiotic bacterial strain comprises a bacterial strain from the genus Bacteroides, Bifidobacterium, Roseburia, Weissella, Enterococcus, Lactococcus, Eubacterium, Butirivibrio, Clostridium group XIVa, or combinations thereof.

Clause 8. The composition of any one of clauses 1 to 7, wherein the probiotic bacterial strain comprises a genetic alteration in one or more genes involved in the phosphotransferase system (PTS).

Clause 9. The composition of clause 8, wherein the one or more genes comprise one or more of a LicT transcriptional anti-terminator, an EiiCBA component of the PTS system, a phospho-β-glucosidase of glycoside hydrolase family 1 (GH1), or any homologous glucosidases and hydrolases,

Clause 10. The composition of any one of clauses 1 to 9, wherein the probiotic bacterial strain comprises a genetic alteration in one or more genes that regulate intracellular hydrolysis of plant glycosides,

Clause 11. The composition of clause 10, wherein the one or more genes that regulate the intracellular hydrolysis of plant glycosides encodes an enzyme that hydrolyzes or phosphorylates the plant glycoside.

Clause 12. The composition of clause 11, wherein the enzyme comprises a plant glycoside hydrolase.

Clause 13. The composition of clause 12, wherein the prebiotic plant glycoside hydrolase comprises one or more phospho-β-glucosidases (P-Bgls), β-glucosidases, or rhamnosidases.

Clause 14. The composition of any one of clauses 1 to 13, wherein the prebiotic plant glycoside comprises an aromatic glycoside, a coumarin glucoside, a stilbenoid glucoside, an aryl β-D-glucoside, a resveratrol glucoside derivative, a flavonol, a phenolic, a polyphenolic, or combinations thereof.

Clause 15. The composition of any one of clauses 1 to 14, wherein the prebiotic plant glycoside comprises a glucoside, a fructoside, a rhamnoside, a xyloside, an arabinopyranoside, a glucuronide, or combinations thereof.

Clause 16. The composition of any one of clauses 1 to 15, wherein the prebiotic plant glycoside comprises a mono- or di-glucoside anomerically substituted with a single or double aromatic ring system.

Clause 17. The composition of any one of clauses 1 to 16, wherein the prebiotic plant glycoside is one or more of Amygdalin, Arbutin, Aucubin, Daidzin, Esculin, Fraxin, Isoquercetin, Polydatin, Rutin hydrate, Salicin, Sinigrin hydrate, Vanilin 4-O-β-glucoside, or glucoside derivatives thereof.

Clause 18. The composition of any one of clauses 1 to 17, wherein the prebiotic plant glycoside is Polydatin.

Clause 19. The composition of any one of clauses 1 to 18, wherein the physiologically acceptable excipient comprises one or more of cellulose, microcrystalline cellulose, mannitol, glucose, sucrose, trehalose, xylose, skim milk, milk powder, polyvinylpyrrolidone, tragacanth, acacia, starch, alginic acid, gelatin, dibasic calcium phosphate, stearic acid, croscarmellose, silica, polyethylene glycol, hemicellulose, pectin, amylose, amylopectin, xylan, arabinogalactan, polyvinylpyrrolidone, and combinations thereof.

Clause 20. A nutritional supplement comprising the composition of any one of clauses 1 to 19.

Clause 21. A method for providing a dietary supplement to a subject, the method comprising administering to the subject the composition of any one of clauses 1 to 19 or the nutritional supplement of clause 20.

Clause 22. A method of supplementing a fermented dairy product, the method comprising mixing the composition of any one of clauses 1 to 19 or the nutritional supplement of clause 20 with a fermented dairy product.

Clause 23. A method of treating a condition in a subject in need thereof, the method comprising administering the composition of any one of clauses 1 to 19 or the nutritional supplement of clause 20 to the subject, thereby treating the condition.

Clause 24. The method of clause 23, wherein the condition is one or more of obesity, cardiovascular disease, metabolic syndrome, cancer, autoimmune disease, inflammatory disorder, digestive system disorder, digestive system-related disorder, or combinations thereof.

Clause 25. The method of clause 23, wherein the composition or nutritional supplement is administered in the form of a tablet, pill, capsule, powder, lozenge, or suppository.

Clause 26. The method of clause 23, wherein treating the subject comprises the probiotic bacterial strain internalizing the prebiotic plant glycoside, converting the prebiotic plant glycoside into a bioactive aglycone, or derivative thereof, and releasing the bioactive aglycone, wherein the bioactive aglycone is absorbed by the subject.

What is claimed is:

1. A composition comprising:
a probiotic bacterial strain;
a prebiotic plant glycoside; and
a physiologically acceptable carrier and/or excipient;
wherein the probiotic bacterial strain is capable of converting e prebiotic plant glycoside into a bioactive aglycone, or derivative thereof.

2. The composition of claim wherein the probiotic bacterial strain comprises a bacterial species from the genus Lactobacillus.

3. The composition of claim 2, wherein the bacterial species is L. acidophilus, L. amylovorus, L. animalis, L. crispatus, L. ferinentum, L. gasserii, L. helveticus, L. intestinalis, jensenii, L. johnsonii, L. plantarum, L. reuteri, L. rhamnosus, and combinations thereof.

4. The composition of claim 3, wherein the bacterial strain is selected from the group consisting of L. acidophilus LA-1, L. acidophilus NCFM, L. amylovorus (ATCC 33620, DSM 20531), L. animalis (DSM 20602), L. crispatus (ATCC 33820, DSM 20584), L. firmentum (ATCC 14931), L. gasseri (ATCC 33323), L. helveticus CNRZ32, L. intestinalis Th4 (ATCC 49335, DSM 6629), L. jensenii (ATCC 25258, 62G, DSM 20557), L. johnsonii (ATCC 33200), L. plantarum sp. plantarum (ATCC 14917, LA70), L. reuteri (ATCC 23272, DSM 20016), L. rhamnosus GG (ATCC 53103), and combinations thereof.

5. The composition of claim 4, wherein the bacterial strain is L. acidophilus NCFM.

6. The composition of claim 1, further comprising at least a second probiotic bacterial strain that is not a bacterial species from the genus Lactobacillus.

7. The composition of claim 6, wherein the at least second probiotic bacterial strain comprises a bacterial strain from the genus Bacteroides, Bifidobacterium, Roseburia, Weissella, Enterococcus, Lactococcus, Eubacterium, Butirivibrio, Clostridium group XIVa, or combinations thereof.

8. The composition of claim wherein the probiotic bacterial strain comprises a genetic alteration in one or more genes involved in the phosphotransferase system (PTS).

9. The composition of claim 8, wherein the one or more genes comprise one or more of a LicT transcriptional anti-terminator, an EIICBA component of the PTS system, a phospho-β-glucosidase of glycoside hydrolase family 1 (GH1), or any homologous glycosidases and hydrolases.

10. The composition of claim 1, wherein the probiotic bacterial strain comprises a genetic alteration in one or more genes that regulate intracellular hydrolysis of plant glycosides.

11. The composition of claim 10, wherein the one or more genes that regulate the intracellular hydrolysis of plant glycosides encodes an enzyme that hydrolyzes or phosphorylates the plant glycoside.

12. The composition of claim 11, wherein the enzyme comprises a plant glycoside hydrolase.

13. The composition of claim 12, wherein the prebiotic plant glycoside hydrolase comprises one or more phospho-β-glucosidases (P-Bgls), β-glucosidases, or rhamnosidases.

14. The composition of claim 1, wherein the prebiotic plant glycoside comprises an aromatic glycoside, a coumarin glucoside, a stilbenoid glucoside, an aryl β-D-glucoside, a resveratrol glucoside derivative, a flavonol, a phenolic, a polyphenolic, or combinations thereof.

15. The composition of claim 1, wherein the prebiotic plant glycoside comprises a glucoside, a fructoside, a rhamnoside, a xyloside, an arabinopyranoside, a glucuronide, or combinations thereof.

16. The composition of claim 1, wherein the prebiotic plant glycoside comprises a mono- or di-glucoside anomerically substituted with a single or double aromatic ring system.

17. The composition of claim 1, wherein the prebiotic plant glycoside is one or more of Amygdalin, Arbutin, Aucubin, Daidzin, Esculin, Fraxin, Isoquercetin, Polydatin, Rutin hydrate, Salicin, Sinigrin hydrate, Vanilin 4-O-β-glucoside, or glucoside derivatives thereof.

18. The composition of claim 1, wherein the prebiotic plant glycoside is Polydatin.

19. The composition of claim 1, wherein the physiologically acceptable excipient comprises one or more of cellulose, microcrystalline cellulose, mannitol, glucose, sucrose, trehalose, xylose, skim milk, milk powder, polyvinylpyrrolidone, tragacanth, acacia, starch, alginic acid, gelatin, dibasic calcium phosphate, stearic acid, croscarmellose, silica, polyethylene glycol, hemicellulose, pectin, amylose, amylopectin, xylan, arabinogalactan, polyvinylpyrrolidone, and combinations thereof.

20. A nutritional supplement comprising the composition of claim 1.

21. A method for providing a dietary supplement to a subject, the method comprising administering to the subject the composition of claim 1.

22. A method of supplementing a fermented dairy product, the method comprising mixing the composition of claim 1.

23. A method of treating a condition in a subject in need thereof, the method comprising administering the composition of claim 1 to the subject, thereby treating the condition.

24. The method of claim 23, wherein the condition is one or more of obesity, cardiovascular disease, metabolic syndrome, cancer, autoimmune disease, inflammatory disorder, digestive system disorder, digestive system-related disorder, or combinations thereof.

25. The method of claim 23, wherein the composition or nutritional supplement is administered in the form of a tablet, pill, capsule, powder, lozenge, or suppository.

26. The method of claim 23, wherein treating the subject comprises:
the probiotic bacterial strain internalizing the prebiotic plant glycoside, converting the prebiotic plant glycoside into a bioactive aglycone, or derivative thereof and releasing the bioactive aglycone;
wherein the bioactive aglycone is absorbed by the subject.
